(19) United States
(12) Patent Application Publication
(10) Pub. No.: US 2015/0065416 A1
(43) Pub. Date: Mar. 5, 2015

(54) NOVEL DRUG COMBINATION

(71) Applicant: FAB PHARMA SAS, Paris (FR)

(72) Inventors: Coralie Mouzé, Paris (FR); Vincent Gerusz, Paris (FR); Alexis Denis, Paris (FR)

(21) Appl. No.: 14/472,866

(22) Filed: Aug. 29, 2014

Related U.S. Application Data
(60) Provisional application No. 61/871,590, filed on Aug. 29, 2013.
(60) Provisional application No. 61/874,516, filed on Sep. 6, 2013.

(51) Int. Cl. A61K 31/166; A61K 31/7036; A61K 31/43; A61K 31/65; A61K 31/496; A61K 31/407; A61K 38/12

(52) U.S. Cl. CPC A61K 31/166; A61K 31/407; A61K 31/7036; A61K 38/12; A61K 31/65; A61K 31/496; A61K 31/43
USPC 514/2.8; 514/210.13; 514/40; 514/154; 514/192

(57) ABSTRACT

There is provided inter alia a method of treating microbial infection comprising administering to a subject in need thereof a therapeutically effective amount of:
(a) 4-(4-ethyl-5-fluoro-2-hydroxyphenoxy)-3-fluorobenzamide; and
(b) an antibacterial agent selected from the group consisting of carbapenems, aminoglycosides, polymixins, glycylcyclines, rifampifin and sulbactam.

### FIELD OF THE INVENTION

The present invention relates to methods of treating microbial infections, in particular infections caused by Acinetobacter baumannii, using novel combinations of known antibacterial agents. The present invention also relates to the use of such novel combinations as antibacterial medicaments and to pharmaceutical combinations containing such novel combinations.

### BACKGROUND OF THE INVENTION

The emergence of antibiotic-resistant pathogens has become a serious worldwide healthcare problem. Indeed, some infections are now caused by multi-drug resistant organisms that are no longer responsive to currently available treatments. There is therefore an immediate need for new antibacterial agents and formulations with a novel mode of action.

The bacterial fatty acid biosynthesis (FASII system) has generated substantial interest for the development of novel antibacterial and antiparasitic agents (Rock et al. J. Biol. Chem. 2006, 281, 17541; Wright and Reynolds Curr. Opin. Microbiol. 2007, 10, 447). The organization of components in the bacterial fatty acid biosynthesis pathway based on discrete enzymes in some bacteria and parasites is fundamentally different from the multifunctional FASI system found in mammals, therefore allowing good prospects of selective inhibition. The overall high degree of conservation in many enzymes of the bacterial FASII system should also allow the development of broader-spectrum antibacterial and antiparasitic agents.

Among all the monofunctional enzymes of the bacterial FASII system, FabI represents the enoyl-ACP reductase responsible of the last step of the fatty acid biosynthetic elongation cycle. Using the cofactor NAD(P)H as a hydride source, FabI reduces the double bond in the trans-2-enoyl-ACP intermediate to the corresponding acyl-ACP product. This enzyme has been shown to constitute an essential target in major pathogens such as E. coli (Heath et al. J. Biol. Chem. 1995, 270, 26538; Bergler et al. Eur. J. Biochem. 1996, 242, 689) and S. aureus (Heath et al. J. Biol. Chem. 2000, 275, 4654). However, other isoforms have been isolated such as FabK from S. pneumoniae (Heath et al. Nature 2000, 406, 145) and FabI from B. subtilis (Heath et al. J. Biol. Chem. 2000, 275, 40128). Although FabK is structurally and mechanistically unrelated to FabI (Marrakchi et al. Biochem J. 2003, 370, 1055), the similarity of FabI with FabI (B. subtilis), InhA (M. tuberculosis) and PfENR(P. falciparum) still offers opportunities of interesting activity spectra (Heath et al. Prog. Lipid Res. 2001, 40, 467).

Several FabI inhibitors have already been reported in the literature (Tonge et al. Acc. Chem. Res. 2008, 41, 11). Some of them such as diazaborines (Baldock et al. Science 1996, 274, 2107) and isoniazid in its activated form (Tonge et al. Proc. Natl. Acad. Sci. U.S.A. 2003, 100, 13881) act by covalently modifying the cofactor NAD+. However some drawbacks are associated with these products. Diazaborines are only used experimentally because of their inherent toxicity (Baldock et al. Biochem. Pharmacol. 1998, 55, 1541) while isoniazid is a prodrug restricted to the treatment of susceptible tuberculosis. The fact that isoniazid requires activation by hydrogen-peroxide inducible enzymes (Schultz et al. J. Am. Chem. Soc. 1995, 117, 5009) enhances the possibilities of resistance by lack of activation or increased detoxification (Rosner et al. Antimicrob. Agents Chemother. 1993, 37, 2251 and ibid 1994, 38, 1829).

Other inhibitors act by interacting noncovalently with the enzyme-cofactor complex. For instance triclosan, a widely used consumer goods preservative with broad spectrum antimicrobial activity, has been found to be a reversible, tight-binding inhibitor of E. coli FabI (Ward et al. Biochemistry 1999, 38, 12514). Intravenous toxicology studies on this compound indicated a LD50 on rats of 29 mg/kg clearly ruling out intravenous injection (Lyman et al. Ind. Med. Surg. 1969, 38, 42). Derivatives based on the 2-hydroxydiphenyl ether core of triclosan have been reported (Tonge et al. J. Med. Chem. 2004, 47, 509, ACS Chem. Biol. 2006, 1, 43 and Bioorg. Med. Chem. Lett. 2008, 18, 3029; Surolia et al. Bioorg. Med. Chem. 2006, 14, 8086 and ibid 2008, 16, 5536; Freundlich et al. J. Biol. Chem. 2007, 282, 25436) as well as other inhibitors based on various classes of high throughput screening derived templates (Seefeld et al. Bioorg. Med. Chem. Lett. 2001, 11, 2241 and J. Med. Chem. 2003, 46, 1627; Heerding et al. Bioorg. Med. Chem. Lett. 2001, 11, 2061; Miller et al. J. Med. Chem. 2002, 45, 3246; Payne et al. Antimicrob. Agents Chemother. 2002, 46, 3118; Sacchettini et al. J. Biol. Chem. 2003, 278, 20851; Moir et al. Antimicrob. Agents Chemother. 2004, 48, 1541; Montellano et al. J. Med. Chem. 2006, 49, 6308; Kwak et al. Int. J. Antimicro. Ag. 2007, 30, 446; Lee et al. Antimicrob. Agents Chemother. 2007, 51, 2591; Kitagawa et al. J. Med. Chem. 2007, 50, 4710, Bioorg. Med. Chem. 2007, 15, 1106 and Bioorg. Med. Chem. Lett. 2007, 17, 4982; Takahata et al. J. Antibiot. 2007, 60, 123; Kozikowski et al. Bioorg. Med. Chem. Lett. 2008, 18, 3565), nevertheless none of these inhibitors has succeeded yet as a drug. Interestingly, some classes of these inhibitors display activity on both FabI and FabK: predominantly FabK for the dual compounds based on phenylimidazole derivatives of 4-pyridones (Kitagawa et al. J. Med. Chem. 2007, 50, 4710), predominantly FabI for the indole derivatives (Payne et al. Antimicrob. Agents Chemother. 2002, 46, 3118; Seefeld et al. J. Med. Chem. 2003, 46, 1627). However, the moderate activity on the second enzyme might prove to be a drawback for such compounds as it may lead to an increase of resistance mechanisms due to the added selection pressure (Tonge et al. Acc. Chem. Res. 2008, 41, 11).

Despite the attractiveness of FabI as an antibacterial/antiparasitic target, however, it is still largely unexploited at this time.

Acinetobacter baumannii (A. baumannii) is a pathogen which has extraordinary ability to acquire resistance to almost all groups of antibiotics and has the ability to survive for extended periods on environmental surfaces. Infection caused by A. baumannii has become a great concern in hospital settings due to high associated mortality rates, reported to be as high as 50% in some intensive care units (Montero et al. Antimicrobial Agents and Chemotherapy 2002, 1946-1952). Most A. baumannii strains isolated in hospitals today are highly resistant to modern noncarbapenem β-lactams, aminoglycosides and fluoroquinolones. Imipenem (a carbapenem antibiotic) used to be considered the “gold standard” therapy for severe infections, but many countries have reported growing resistance to carbapenems.

Thus, there is a pressing need to develop new strategies for treatments that are effective against antibiotic resistant bacterial strains, in particular for the treatment of infection by A. baumannii.

International application WO 2007/135562 (Mutabilis SA) describes a series of hydroxyphenyl derivatives that display a selective spectrum of activity on species containing FabI and related targets, in contrast to Triclosan. A specific hydroxyphenyl compound of formula (I) is described in international application WO 2011/026529 (FAB Pharma SAS):

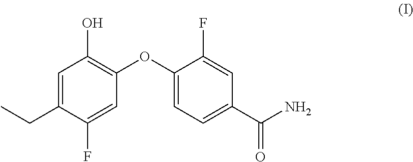 [SMILES: CCc1cc(O)c(Oc2ccc(C(N)=O)cc2F)cc1F]

The compound of formula (I) (referred to herein as “compound (I)”) is known chemically as 4-(4-ethyl-5-fluoro-2-hydroxyphenoxy)-3-fluorobenzamide and has been found to be highly active in vitro against pathogenic methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains. Furthermore, compound (I) is also active in vivo in a murine model against MSSA, MRSA and VISA infections. Compound (I) has been evaluated in healthy human volunteers in a Phase I study and was reported to demonstrate ex vivo bactericidal activity against S. aureus.

It is an object of the present invention to provide a novel combination therapy comprising compound (I) and another known antibacterial agent. Suitably, the combination will have improved antibacterial characteristics compared with compound (I) and the known antibacterial agent, individually. In particular, it is an object of the present invention to provide an effective combination regimen for treating infection caused by Acinetobacter baumannii.

### SUMMARY OF THE INVENTION

The present inventors have discovered that combinations of 4-(4-ethyl-5-fluoro-2-hydroxyphenoxy)-3-fluorobenzamide (compound (I)) with certain known antibacterial agents (or classes of agents) exhibit improved anti-microbial activity, compared with 4-(4-ethyl-5-fluoro-2-hydroxyphenoxy)-3-fluorobenzamide alone, or the known antibacterial agent alone.

Thus, in a first aspect, the present invention provides a method of treating microbial infection comprising administering to a subject in need thereof a therapeutically effective amount of:

(a) 4-(4-ethyl-5-fluoro-2-hydroxyphenoxy)-3-fluorobenzamide; and

(b) an antibacterial agent selected from the group consisting of carbapenems, aminoglycosides, polymixins, glycylcyclines, rifampicin and sulbactam.

In second aspect, the present invention provides 4-(4-ethyl-5-fluoro-2-hydroxyphenoxy)-3-fluorobenzamide for use in treating microbial infection in a subject, in a therapeutic regimen comprising said 4-(4-ethyl-5-fluoro-2-hydroxyphenoxy)-3-fluorobenzamide in combination with an antibacterial agent selected from the group consisting of carbapenems, aminoglycosides, polymixins, glycylcyclines, rifampicin and sulbactam.

In a third aspect, the present invention provides an antibacterial agent selected from the group consisting of carbapenems, aminoglycosides, polymixins, glycylcyclines, rifampicin and sulbactam for use in treating microbial infection in a subject, in a therapeutic regimen comprising said antibacterial agent in combination with 4-(4-ethyl-5-fluoro-2-hydroxyphenoxy)-3-fluorobenzamide.

In a fourth aspect, the present invention provides the use of 4-(4-ethyl-5-fluoro-2-hydroxyphenoxy)-3-fluorobenzamide and an antibacterial agent selected from the group consisting of carbapenems, aminoglycosides, polymixins, glycylcyclines, rifampicin and sulbactam in the manufacture of a medicament for use in treating microbial infection.

In a fifth aspect, the present invention provides a pharmaceutical composition comprising 4-(4-ethyl-5-fluoro-2-hydroxyphenoxy)-3-fluorobenzamide and an antibacterial agent selected from the group consisting of carbapenems, aminoglycosides, polymixins, glycylcyclines, rifampicin and sulbactam.

As shown in the Examples, novel combinations of compound (I) and an additional antibacterial agent can, both in vitro and in vivo, provide enhanced antibacterial activity against A. baumannii compared with compound (I) alone or the additional antibacterial agent alone. Thus, the present invention provides novel combination therapies which in at least some embodiments provide an improved method of treating microbial infection, in particular antibiotic-resistant microbial infection, such as antibiotic-resistant A. baumannii.

### BRIEF DESCRIPTION OF THE FIGURES

FIG. 1 shows minimum inhibitory concentration (MIC) distribution for compound (I) against 77 isolates of A. baumannii (Example 2).

FIG. 2 shows MIC distribution for compound (I), amikacin, colistin, gentamicin, meropenem and tobramycin against 77 isolates of A. baumannii (Example 2).

FIG. 3 shows a summary of checkerboard experiments with twenty A. baumannii isolates and combinations of compound (I) with amikacin, colistin, gentamicin, meropenem and tobramycin (Example 3).

FIGS. 4-9 show the bactericidal effects of compound (I)/amikacin combinations against various A. baumannii isolates (Example 4).

FIGS. 10-15 shows the bactericidal effects of compound (I)/meropenem combinations against various A. baumannii isolates (Example 4).

FIGS. 16-27 show the bactericidal effects of compound (I)/colistin combinations against various A. baumannii isolates (Example 4).

FIG. 28 summarises the in vitro time kill kinetics of meropenem, amikacin and compound (I) alone or in combination, against A. baumannii SAN strain (Example 5).

FIG. 29 shows the effect of antibacterial agent therapy on survival 72 hours post infection with A. baumannii for Study 1 of the mouse model (Example 6).

FIG. 30 shows the effect of antibacterial agent therapy on survival 48 hours post infection with A. baumannii for Study 3 of the mouse model (Example 6).

FIG. 31 shows the effect of antibacterial agent therapy on survival 72 hours post infection with A. baumannii for Study la of the mouse model (Example 7).

### DETAILED DESCRIPTION OF THE INVENTION

### 4-(4-Ethyl-5-fluoro-2-hydroxyphenoxy)-3-fluorobenzamide (compound (I))

General processes for synthesising the compound of formula (I) are disclosed in WO 2007/135562, the contents of which is incorporated by reference in its entirety. A specific method for synthesising the compound of formula (I) is set out in Example 1 of the present application, and corresponds to the procedure provided in “Example 1 (Alternative procedure)” of WO 2011/026529, the contents of which is also incorporated by reference in its entirety.

The compound of formula (I) is 4-(4-ethyl-5-fluoro-2-hydroxyphenoxy)-3-fluorobenzamide or a pharmaceutically acceptable salt or solvate thereof.

In the present context, the term “pharmaceutically acceptable salt” is intended to indicate salts which are not harmful to the patient. Such salts include pharmaceutically acceptable acid addition salts, pharmaceutically acceptable metal salts and pharmaceutically acceptable alkaline addition salts. Acid addition salts include salts of inorganic acids as well as organic acids.

Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference in its entirety. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Representative examples of alkaline salts include, for example, sodium, potassium, lithium, calcium, magnesium or ammonium or organic bases such as, for example, methylamine, ethylamine, propylamine, trimethylamine, diethylamine, triethylamine, N,N-dimethylethanolamine, tris(hydroxymethyl)aminomethane, ethanolamine, pyridine, piperidine, piperazine, picoline, dicyclohexylamine, morpholine, benzylamine, procaine, lysine, arginine, histidine, N-methylglucamine.

Examples of solvates include hydrates.

Additional Antibacterial Agents

In the methods and uses of the invention, compound (I) is administered in combination with an antibacterial agent selected from the group consisting of carbapenems, aminoglycosides, polymixins, glycylcyclines, rifampicin and sulbactam. The antibacterial agent selected from the group consisting of carbapenems, aminoglycosides, polymixins, glycylcyclines, rifampicin and sulbactam is referred to herein as the “additional antibacterial agent”.

Carbapenems

Carbapenems are broad spectrum β-lactam antibacterial agents that exhibit antibacterial activity by interfering with bacterial cell wall synthesis by binding to penicillin-binding proteins (PBPs). PBPs are enzymes that catalyse the formation of peptidoglycan in the cell wall of bacteria. Bacterial cell wall formation is a dynamic process with formation and autolysis occurring at the same time. Therefore, when PBP function is inhibited, cell wall formation is interrupted while autolysis continues and the peptidoglycan weakens. Eventually the bacterial cell bursts due to osmotic pressure, leading to bacterial cell death.

Examples of carbapenems include but are not limited to: meropenem, imipenem, ertapenem, doripenem, panipenem and biapenem. In one embodiment, the carbapenem is meropenem or imipenem, suitably meropenem.

Aminoglycosides

Aminoglycosides are broad spectrum antibacterial agents that exhibit antibacterial activity by binding to the ribosomal decoding site, which has the effect of reducing the fidelity of protein synthesis.

Examples of aminoglycosides include but are not limited to amikacin, gentamicin, tobramycin, arbekacin, kanamycin, neomycin, netilmicin, paromomycin, rhodostreptomycin, streptomycin and apramycin. In one embodiment, the aminoglycoside antibacterial agent is amikacin, gentamicin or tobracmycin, suitably amikacin.

Treatment with aminoglycosides can cause side effects including ototoxicity and nephrotoxicity.

Polymixins

There are five different polymixin compounds, polymixins A-E, however only polymixins B and polymixin E are used in clinical practice. Polymixin E is also known as colistin, and is commercially available in cationic form as colistin sulfate, and in anionic form as colistin methanesulfonate sodium (colistimethate sodium (CMS)). In one embodiment, the polymixin antibacterial agent is colistin, suitably colistin methanesulfonate sodium.

Polymixin antibacterial agents target Gram-negative bacteria and exhibit antibacterial activity by binding to the lipopolysaccharide in the outer membrane of the bacteria, disrupting both the outer and inner membranes, leading to leakage of the intracellular contents and bacterial cell death.

The most common adverse effect of colistin treatment is nephrotoxicity (renal toxicity) because the drug is excreted primarily by the kidneys and elevated blood levels may further impair renal function. However, the use of more purified colistin, the use of colisin methanesulfonate sodium instead of colistin sulphate, more adequate dose adjustment according to renal function and significant improvement of ICU monitoring and treatment has resulted in a relatively lower incidence of renal toxicity (Spapen et al., Annals of Intensive Care 2011, 1:14, 1-7). The interaction of colistin with neurons, which have high lipid content, has been associated with the occurrence of peripheral and orofacial paresthesias, vertigo, mental confusion, ataxia and seizures. However, a review by Spapen et al. concluded that neurotoxicity was not a major adverse event accompanying colistin treatment (Annals of Intensive Care 2011, 1:14, 1-7).

Glycylcyclines

Glycylcycline antibacterial agents are broad spectrum antibacterial agents that were developed as tetracycline analogues in order to overcome tetracycline resistance. This structural class of antibacterial agents is bacteriostatic, and act by binding to the bacterial 30S ribosomal subunit and by blocking entry of amino-acyl tRNA molecules into the A site of the ribosome. Amino acid residues are prevented from becoming incorporated into elongating peptide chains, which leads to inhibition of protein synthesis.

The only glycylcycline antibacterial agent presently available for clinical use is tigecycline, which is available only as an injectable formulation. Thus, in one embodiment, the glycylcycline antibacterial agent is tigecycline. According to a review by Noskin, G. A. (Clinical Infectious Diseases 2005, S303-14) tigecycline has shown promise as a monotherapy and its use as part of a combination regimen has not been evaluated.

Rifampicin

Rifampicin is a bactericidal antibacterial agent of the rifamycin group and is typically used to treat Mycobacterium infections including tuberculosis and Hansen's disease. Rifampicin inhibits bacterial DNA-dependent RNA synthesis by inhibiting bacterial DNA-dependent RNA polymerase. The most serious adverse effect of administering rifampicin is hepatotoxicity.

Sulbactam

Sulbactam is a β-lactamase inhibitor which is typically administered in combination with a β-lactam antibiotic. β-lactamases are enzymes which degrade β-lactam antibiotics, therefore when sulbactam binds to the enzyme it prevents degradation of the β-lactam antibiotic.

In one embodiment, the antibacterial agent in combination with compound (I) is selected from the group consisting of meropenem, imipenem, amikacin, gentamicin, tobramycin, colistin, tigecycline, rifampicin and sulbactam and is suitably selected from the group consisting of meropenem, amikacin and colistin.

Microbial Pathogens

As illustrated in the Examples, novel combinations of compound (I) with certain known antibacterial agents have potential utility for treating infection by microbial pathogens.

Thus, in one embodiment, the antibacterial combinations of the present invention can be used to treat microbial infection in humans and animals. Antibacterial combinations of the present invention are particularly useful in forming a therapeutic regimen having a selective spectrum of activity in vitro and in vivo against bacterial strains relying on FabI and related targets.

Such strains encompass Staphylococcus aureus including methicillin-susceptible Staphylococcus aureus (MSSA) and multiresistant strains (such as methicillin-resistant Staphylococcus aureus (MRSA), vancomycin-intermediate Staphylococcus aureus (VISA) and vancomycin-resistant Staphylococcus aureus (VRSA) strains), Acinetobacter strains (such as Acinetobacter baumannii), Bacillus anthracis, Chlamydophila pneumoniae, Escherichia coli, Haemophilus influenzae, Helicobacter pylori, Klebsiella pneumoniae, Neisseria meningitidis and also bacteria such as Mycobacterium tuberculosis carrying homologous FabI enzymes such as InhA or other organisms such as Plasmodium falciparum. Acinetobacter strains include isolates independently resistant to carbapenems, aminoglycosides, polymixins and fluoriquinolones.

In one embodiment, the antibacterial combination of the present invention is used in the treatment of human or animal microbial infection by Acinetobacter, in particular Acinetobacter baumannii.

Acinetobacter

There are five different phylogenetic Acinetobacter groups involved in human infections: A. baumannii (most frequent), A. nosocomialis, A. pitii, A. calcoaceticus, and the fifth group has been only recently identified and has yet to be named (Nemec A., 2013, 9^{th} International Symposium on the Biology of Acinetobacter).

While all Acinetobacter groups can potentially cause human disease, A. baumannii accounts for about 80% of reported Acinetobacter infections (http://www.cdc.gov/HAI/organisms/acinetobacter.html) A. baumannii is an emergent nosocomial pathogen which has also been isolated from soil and water samples in the environment, although infection rarely occurs outside of healthcare settings. Risk factors associated with A. baumannii include prolonged hospitalization, intensive care unit admission, invasive surgical procedures, previous treatment with antibiotics (such as imipenem and levofloxacin), chronic lung disease and alcoholism (Seifert H., 2013, 9^{th} International Symposium on the Biology of Acinetobacter).

Acinetobacter causes a variety of diseases including blood stream infections, urinary tract infections, meningitis, wound infections especially in burns patients (Dijkshoorn L., 2013, 9th International Symposium on the Biology of Acinetobacter), and pneumonia especially in patients on mechanical ventilation. Acinetobacter may also colonize in a patient without causing infections or symptoms, especially in tracheostomy sites or open wounds (http://www.cdc.gov/HAI/organisms/acinetobacter.html). Acinetobacter can be spread by person-to-person contact, airborne transmission, environmental surfaces and medical equipment (Seifert H., 2013, 9^{th} International Symposium on the Biology of Acinetobacter). Acinetobacter spp. were included in EARS-Net (European Antimicrobial Resistance Surveillance Network) for the first time in 2012. According to this report in 2012, A. baumannii infections are most likely to occur in critically ill or otherwise debilitated individuals and it is likely to be a major contributing factor to nosocomial spread, particularly in intensive care units (ICUs). More than half of Acinetobacter spp. isolates reported to EARS-Net in 2012 were resistant to all antibiotic groups included for surveillance. Carbapenem resistance was high, and in most cases combined with resistance to the two other antimicrobial groups under surveillance (fluoroquinolones and aminoglycosides). However, large inter-country variation was observed, with generally higher resistance levels reported from southern Europe than northern Europe. Overall, the most common resistance phenotype was resistance to all three antimicrobial groups, and was present in 51% of the European isolates (http://www.ecdc.europa.eu/en/publications/Publications/antimicrobial-resistance-surveillance-europe-2012.pdf).

In one embodiment the infection by A. baumannii is associated with a disease or disorder selected from the group consisting of a blood stream infection, a urinary tract infection, meningitis, a wound infection (especially in a burns patient) and pneumonia (especially in a patient on mechanical ventilation).

As shown in the checkerboard studies of Example 3, over 70% of the combinations of compound (I) and an additional antibacterial agent as defined herein exhibited additivity and/or synergy, with colistin in particular demonstrating synergy with compound (I). In Example 4, sub-MIC combinations of compound (I) and amikacin and meropenem were found to exhibit bactericidal activity (i.e. time to attain 99.9% bacterial killing, which is defined as a Δ3 log 10 reduction compared to initial inoculum).

In vivo studies (Example 6) using a mouse model of A. baumannii pneumonia demonstrated that administering combinations of compound (I) with amikacin and meropenem increased survival rates compared with administering amikacin and meropenem alone. A combination of compound (I) and colistin demonstrated greater efficacy than compound (I) alone. An in vivo study (Example 7) also using a mouse model of A. baumannii pneumonia demonstrated that administering a combination of compound (I) with meropenem increased survival rates compared with administering compound (I) alone.

Pharmaceutical Composition

The combination of compound (I) and the additional antibacterial agent can be co-formulated as a pharmaceutical composition. Thus, in one embodiment, there is provided a pharmaceutical composition comprising:

(a) 4-(4-ethyl-5-fluoro-2-hydroxyphenoxy)-3-fluorobenzamide (compound (I)); and

(b) an antibacterial agent selected from the group consisting of carbapenems, aminoglycosides, polymixins, glycylcyclines, rifampicin and sulbactam.

Alternatively, compound (I) and the additional antibacterial agent can be formulated as separate pharmaceutical compositions.

Thus, in one embodiment, there is provided a kit of parts comprising:

(a) a pharmaceutical composition comprising 4-(4-ethyl-5-fluoro-2-hydroxyphenoxy)-3-fluorobenzamide; and

(b) a pharmaceutical composition comprising an antibacterial agent selected from the group consisting of carbapenems, aminoglycosides, polymixins, glycylcyclines, rifampicin and sulbactam.

Said kit of parts will typically be accompanied by instructions specifying the co-administration of (a) and (b).

Such pharmaceutical compositions (co-formulated or separate) can be formulated to be administered under oral, topical, parenteral including injectable routes, such as intravenous administration, with individual doses appropriate for the patient to be treated.

Suitably, compound (I) is not administered orally. Suitably, compound (I) is formulated to be administered via parenteral routes. Compound (I) and the additional antibacterial agent will typically be formulated for administration via the same route, thus in one embodiment compound (I) and the additional antibacterial agent are both formulated for administration via parenteral routes. However, compound (I) and the additional antibacterial agent need not be formulated for administration via the same route. For example, compound (I) may be formulated for administration via parenteral routes and the additional antibacterial agent formulated for oral administration.

The compositions according to the invention can be solid, liquid or in the form of a gel/cream and can be present in the pharmaceutical forms commonly used in human medicine, such as for example, plain or sugar-coated tablets, gelatin capsules, granules, suppositories, injectable preparations, ointments, creams, gels; they are prepared according to the customary methods. The active ingredient/s can be incorporated using excipients which are customarily used in these pharmaceutical compositions, such as talc, gum arabic, lactose, starch, magnesium stearate, aqueous or non-aqueous vehicles, fatty substances of animal or vegetable origin, paraffin derivatives, glycols, various wetting agents, dispersants or emulsifiers, preservatives. These compositions can also be present in the form of a powder intended to be dissolved extemporaneously in an appropriate vehicle, for example, non-pyrogenic sterile water.

In one embodiment, the pharmaceutical composition additionally comprises a solubilisation agent.

In one embodiment, the pharmaceutical composition additionally comprises an isotonic agent.

In one embodiment, the pharmaceutical composition additionally comprises a diluent. In a further embodiment, the diluent comprises water, such as QS water.

Modes of Administration

Suitably, compound (I) and the additional antibacterial agent are administered via the same route of administration. Suitably, compound (I) and the additional antibacterial agent are both administered intravenously. As discussed above, compound (I) may be co-formulated with the additional antibacterial agent, or formulated separately. Suitably, compound (I) is formulated separately from the additional antibacterial agent, but both compound (I) and the additional antibacterial agent are administered intravenously.

In the methods and uses of the invention compound (I) and the additional antibacterial agent are administered as a combination, i.e. they are co-administered. The co-administration may occur simultaneously or sequentially.

In one embodiment, compound (I) and the additional antibacterial agent are administered simultaneously. Simultaneous administration can involve the co-formulation of compound (I) and the additional antibacterial agent. Alternatively, compound (I) and the additional antibacterial agent can be formulated separately, but administered to the patient at the same time. Suitably, compound (I) and the additional antibacterial agent will be administered simultaneously, via separate or the same parenteral routes.

In one embodiment, compound (I) and the additional antibacterial agent are administered sequentially. In one embodiment, compound (I) is administered prior to the additional antibacterial agent. In another embodiment, the additional antibacterial agent is administered prior to compound (I). In a therapeutic regimen comprising sequential administration either:
1. compound (I) is administered to a subject, then after a period of time the additional antibacterial agent is administered to the subject; or
2. the additional antibacterial agent is administered to a subject, then after a period of time compound (I) is administered to the subject.

The period of time between administration of the two agents (i.e. the period of time referred to at point 1. or 2., above) may be up to 12 hours, for example up to 11 hours, up to 10 hours, up to 9 hours, up to 8 hours, up to 7 hours, up to 6 hours, up to 5 hours, up to 4 hours, up to 3 hours, up to 2 hours, up to 1 hour, up to 50 minutes, up to 45 minutes, up to 30 minutes, up to 25 minutes, up to 20 minutes, up to 15 minutes, up to 10 minutes, up to 9 minutes, up to 8 minutes, up to 7 minutes, up to 6 minutes, up to 5 minutes, up to 4 minutes, up to 3 minutes, up to 2 minutes, up to 1 minutes or up to 30 seconds.

The combination of compound (I) and the additional antibacterial agent (whether via simultaneous or sequential administration) may be administered as a daily dosage, or may be administered at more frequent intervals such as twice daily. Treatment should be continued for as long as required to receive the benefit of the invention.

The absolute and relative dosages of compound (I) and the additional antibacterial agent will depend on the nature of the additional antibacterial agent.

In one embodiment, the dosage of compound (I) in the combination will be lower than the MIC of compound (I) when administered as a monotherapy.

In one embodiment, the dosage of the additional antibacterial agent in the combination will be lower than the MIC of the additional antibacterial agent when administered as a monotherapy.

In one embodiment, the individual dosages of compound (I) and the additional antibacterial agent in the combination will both be lower than their respective MICs when administered as a monotherapy.

Typically, the dosage of compound (I) in the combination will be between about 1000 mg/kg and about 0.1 mg/kg, such as between about 800 mg/kg and about 1 mg/kg, between about 500 mg/kg and about 10 mg/kg or between about 300 mg/kg and about 100 mg/kg, such as about 200 mg/kg.

If the additional antibacterial agent is a carbapenem, typically the dosage of carbapenem will be between about 1000 mg/kg and about 0.1 mg/kg, such as between about 800 mg/kg and about 1 mg/kg, between about 500 mg/kg and about 10 mg/kg, or between about 150 mg/kg and about 10 mg/kg, such as about 50 mg/kg.

Thus, in one embodiment an adult dosage regimen comprises a dose of 200 mg/kg of compound (I) and 50 mg/kg of a carbapenem, dosed simultaneously or sequentially.

If the additional antibacterial agent is an aminoglycoside such as amikacin, typically the dosage of aminoglycoside will be between about 1000 mg/kg and about 0.1 mg/kg, such as between about 800 mg/kg and about 1 mg/kg, between about 500 mg/kg and about 5 mg/kg or between about 50 mg/kg and about 5 mg/kg, such as about 18 mg/kg.

Thus, in one embodiment an adult dosage regimen comprises a dose of 200 mg/kg of compound (I) and 18 mg/kg of an aminoglycoside, dosed simultaneously or sequentially.

If the additional antibacterial agent is a polymixin such as colistimethate sodium, typically the dosage of polymixin will be between about 1000 mg/kg and about 0.1 mg/kg, such as between about 800 mg/kg and about 0.1 mg/kg, between about 500 mg/kg and about 0.5 mg/kg, between about 50 mg/kg and about 0.5 mg/kg, or between about 10 mg/kg and about 1 mg/kg, such as about 5 mg/kg.

Thus, in one embodiment an adult dosage regimen comprises a dose of 200 mg/kg of compound (I) and 5 mg/kg of polymixin, dosed simultaneously or sequentially.

If the additional antibacterial agent is a glycylcycline such as tigecycline, typically the dosage of glycylcycline will be between about 1000 mg/kg and about 0.1 mg/kg, such as between about 800 mg/kg and about 0.1 mg/kg, between about 500 mg/kg and about 0.1 mg/kg, or between about 300 mg/kg and about 0.1 mg/kg, such as about 1-2 mg/kg.

Thus, in one embodiment an adult dosage regimen comprises a dose of 200 mg/kg of compound (I) and 2 mg/kg of glycylcycline, dosed simultaneously or sequentially.

If the additional antibacterial agent is rifampicin, typically the dosage of rifampicin will be between about 1000 mg/kg and 0.1 mg/kg, such as between about 800 mg/kg and 1 mg/kg, or between about 600 mg/kg and about 1 mg/kg, such as about 20 mg/kg.

Thus, in one embodiment an adult dosage regimen comprises a dose of 200 mg/kg of compound (I) and 20 mg/kg of rifampicin, dosed simultaneously or sequentially.

If the additional antibacterial agent is sulbactam, typically the dosage of sulbactam will be between about 300 mg/kg and about 1 mg/kg, such as between about 150 mg/kg and about 1 mg/kg, or between about 100 mg/kg and about 10 mg/kg, such as about 50 mg/kg.

Thus, in one embodiment an adult dosage regimen comprises a dose of 200 mg/kg of compound (I) and 50 mg/kg of sulbactam, dosed simultaneously or sequentially.

### Abbreviations

I.P. intra peritoneal

MIC minimum inhibitory concentration*

CFU colony-forming unit

FIC fractional inhibitory concentration

AMK amikacin

AMK-R amikacin-resistant

MPM meropenem

MPM-R meropenem-resistant

COL colistin

COL-R colistin-resistant

Sus susceptible

PVP polyvinylpyrrolidone

I.V. intravenous

TSA trypticase soy agar

*Minimum inhibitory concentration as used herein refers to the lowest concentration of an antibacterial agent that will inhibit the visible growth of a bacterial strain after overnight incubation.

### EXAMPLES

### Materials

Clinical Isolates—In Vitro Examples 2, 3 & 4

In the antimicrobial susceptibility testing 77 isolates of A. baumannii (from 2010) in the following sub-groups were tested:
22 isolates generally susceptible to antimicrobial agents (“Sus”)
20 carbapenem-resistant isolates (e.g. “MPM-R”)
20 aminoglycoside-resistant isolates (e.g. “AMK-R”)
15 colistin-resistant isolates. (e.g. “COL-R”)

Checkerboard interaction studies were performed using 5 isolates from each of the sub-groups and time kill studies were performed using 3 isolates from each sub-group.

Antimicrobial Agents—In Vitro Examples 2, 3 & 4

Compound (I) was prepared in-house according to the method described in Example 1. Amikacin, tobramycin, meropenem, colistin and gentamicin were all obtained from Sigma.

General Procedures

Individual Antimicrobial Susceptibility Testing

### Example 2

Minimum inhibitory concentration (MIC) endpoints were determined by broth microdilution according to CLSI guidelines (CLSI, M7-A8 & M100-S22, 2012). Panels were prepared at IHMA using cation-adjusted Mueller-Hinton broth. Colonies were taken directly from a second-pass culture plate and prepared to a suspension equivalent of the 0.5 McFarland standard using normal saline. Inoculation of the MIC plates took place within 15 minutes after adjustment of the inoculum suspension turbidity. The panels were incubated at 35° C. for 20-24 hours before reading the MIC endpoints.

Checkerboard Testing (Compound (I)/Antibacterial Agent Interaction)

Checkerboards (20 isolates, 5 per sub-group) were prepared so as to contain multiple concentrations of compound (I) with different additional antibacterial agents (amikacin, gentamicin, colistin, meropenem and tobramycin) as follows:

Serial two-fold dilutions of the antimicrobial agents alone or in combination in wells of 96 well microtiter plates were inoculated with the appropriate inoculum so that each well contained approximately 4-5×10⁴ CFU/ml. Plates were incubated at 35° C. for 16-20 hours. Combination concentrations were tested between 0.25×MIC and 2×MIC. Growth and sterility controls were also included in each plate.

Synergy, indifference and antagonism were calculated based upon the use of the fractional inhibitory concentration (FIC) indices as described by Pillai, Moellering & Eliopoulos (2005) whereby:

Synergism=FIC index of ≦0.5 Indifference=FIC index >1 but ≦4 Additivity >0.5 to 1 Antagonism=FIC index of >4

Time Kill Study

### Example 4

A total of 12 isolates were tested (three per sub-group either susceptible, meropenem-resistant, amikacin-resistant or colistin-resistant). Two combinations were tested per isolate. Time-kill kinetics were performed by testing compound (I) and an additional antibacterial agent (amikacin, colistin and meropenem) individually at 1× and 4× the corresponding MIC of each antibacterial agent. In addition, a second set of experiments involved the testing of compound (I)/additional antibacterial agent combinations of 0.25×/0.25×, 0.5×/0.5×, 1×/1×, and 4×/4×MIC. Samples were taken at 0, 2, 4, 8 and 24 hours in order to determine colony forming units (CFU/mL). Bactericidal activity is defined by a reduction of the inoculum by 3 log 10 compared to initial inoculum at a defined time point (CLSI M26A, 1999).

### Example 1

### Preparation of 4-(4-ethyl-5-fluoro-2-hydroxyphenoxy)-3-fluorobenzamide (compound (I))

Compound (I) may be prepared using the procedures outlined in international application WO 2011/026529. The procedure corresponding to “Example 1 (Alternative Procedure)” of WO 2011/026529 is outlined below:

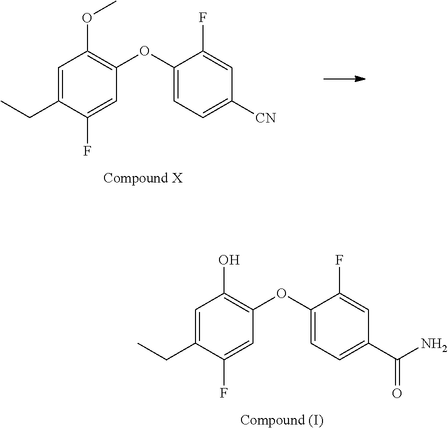 [SMILES: CCc1cc(OC)c(Oc2ccc(C#N)cc2F)cc1F ; CCc1cc(O)c(Oc2ccc(C(N)=O)cc2F)cc1F]

Compound X (disclosed as Intermediate 3 in WO 2011/026529) in acetonitrile was partially distilled under atmospheric pressure to 6.4 residual volumes. 7 volumes of acetic acid were then added and the solution was distilled under atmospheric pressure to 6.4 residual volumes. One additional volume of acetic acid was added and the solution was distilled again under atmospheric pressure to 6.4 residual volumes. Sulphuric acid (6 volumes total) was added and the reaction mixture was stirred at 120° C. for 5 hours.

Once the reaction was complete, the temperature was lowered to 20° C., dichloromethane (10 volumes) and water (8 volumes) were added. At this temperature, Clarcel® (0.5 parts) and charcoal (0.5 parts) were also added and the resulting mixture was stirred for 30 min. The mixture was filtered and the intermediate cake washed three times with 2 volumes of dichloromethane each time. The resulting phases were separated and the aqueous phase was back extracted twice with 3 volumes of dichloromethane. The combined organic phases were partially distilled under atmospheric pressure to 14.5 residual volumes and methylcyclohexane (22 volumes) was added at 37° C.±2° C. To this solution was added sodium bicarbonate (10%) (1 volume). Instantaneous crystallization was observed. The slurry was cooled down to 0° C., filtered, washed twice with 2 volumes of methylcyclohexane at RT and dried at 40° C. under vacuum to afford the crude 4-(4-ethyl-5-fluoro-2-hydroxyphenoxy)-3-fluorobenzamide.

The crude 4-(4-ethyl-5-fluoro-2-hydroxyphenoxy)-3-fluorobenzamide was dissolved in 7 volumes of isopropanol and 1 volume of water at 60° C. to carry out a filtration on a zetacarbon cartridge. The cartridge was then washed twice with 1 volume of isopropanol. Water (12.5 volumes) was added to this solution and the mixture was cooled down at 10° C./h to 5° C. The product was filtered, washed twice with water (2 volumes each time) at RT and dried under vacuum at 70° C. to afford the pure 4-(4-ethyl-5-fluoro-2-hydroxyphenoxy)-3-fluorobenzamide (title compound) in 36.5% overall yield in 99.6% HPLC purity.

### Example 2

### Individual Antimicrobial Susceptibility Testing

Testing was carried out as described in the General Procedures to determine minimum inhibitory concentration (MIC) end points for compound (I), amikacin, colistin, gentamicin, meropenem and tobramycin, for 77 A. baumannii isolates. Under the “MIC (μg/ml)” heading, the first column (Range) indicates the MIC end points for the particular number (N) of isolates tested, the second column (50%) provides an estimate of the concentration that inhibits 50% (MIC50) of bacterial isolates tested, and the third column (90%) provides an estimate of the concentration that inhibits 90% of the bacterial isolates tested.

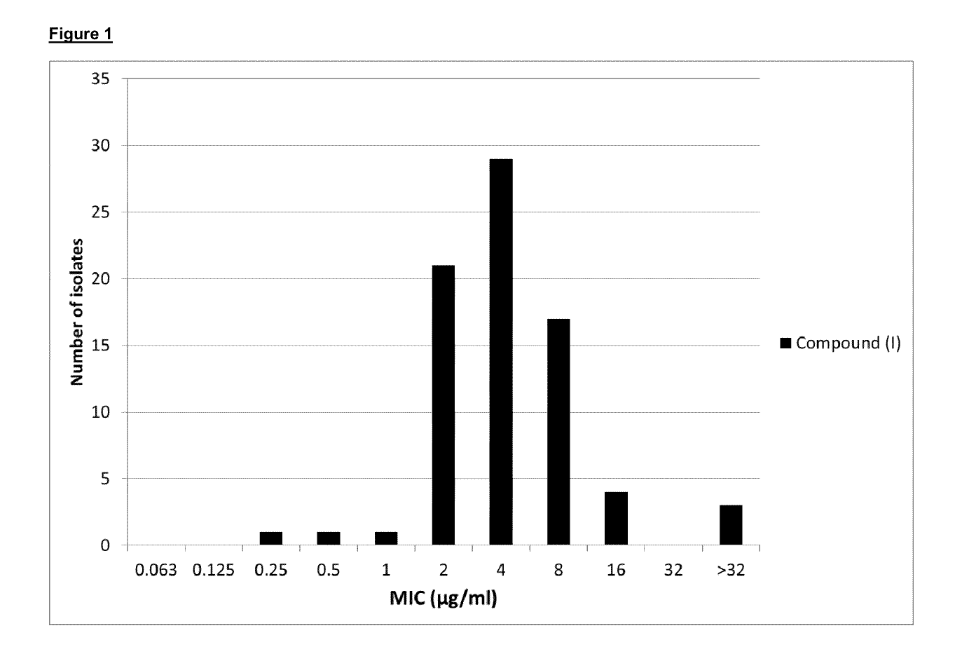

Compound (I) exhibited good activity against the majority of the 77 isolates tested in this study with MICs ranging from 0.5-8,2-8, 0.25->32 and 2->32 pg/ml against susceptible, amikacin-R, colistin-R and meropenem-R isolates respectively. Although the overall MIC range was quite broad, the majority of Compound (I) MICs were in the range of 2-8 μg/ml. The mode MIC was 4 μg/ml (FIG. 1).

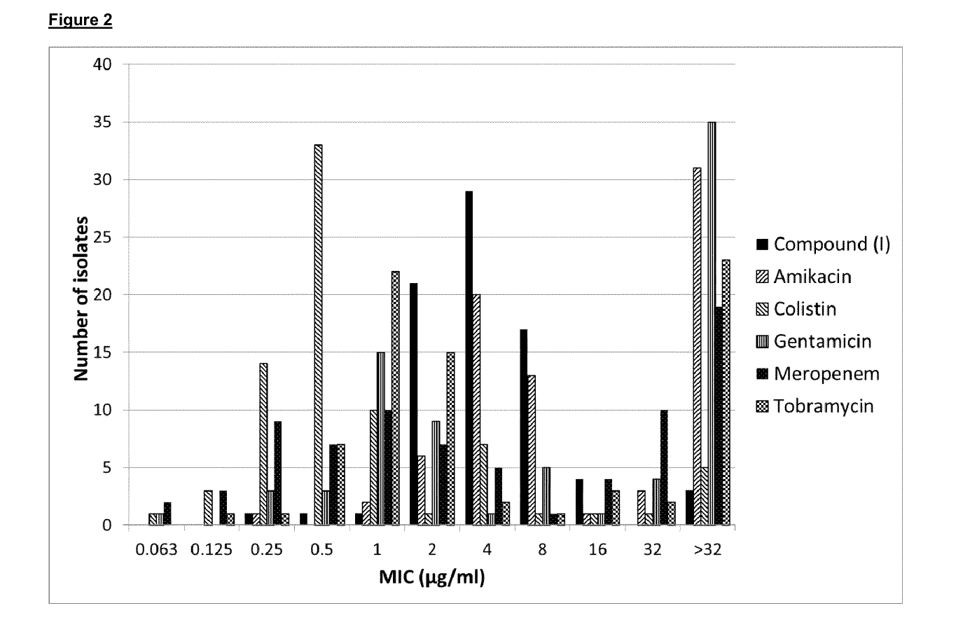

The MIC distributions for compound (I), amikacin, colistin, gentamicin, meropenem and tobramycin are shown in FIG. 2. This Figure shows that colistin had the lowest mode MIC, followed by tobramycin and compound (I). The isolates resistant to amikacin, gentamicin, tobramycin and meropenem can be seen to the right of FIG. 2 (MIC >32 pg/ml). Summary MIC data are given in Table 1.

TABLE 1
Summary of MIC data
Antibac-

| terial | | | MIC (μg/ml) | | |
|---|---|---|---|---|---|
| agent | Subset | N* | Range | 50% | 90% |
| Com- | All | 77 | 0.25−>32 | 4 | 8 |
| pound | Amikacin-resistant | 20 | 2-8 | 4 | 8 |
| (I) | Colistin-resistant | 15 | 0.25−>32 | 4 | >32 |
| | Meropenem-resistant | 20 | 2−>32 | 8 | 8 |
| | Susceptible | 22 | 0.5-8 | 4 | 8 |
| Amikacin | All | 77 | 0.25−>128 | 8 | >128 |
| | Amikacin-resistant | 20 | 128−>128 | >128 | >128 |
| | Colistin-resistant | 15 | 2−>128 | 64 | >128 |
| | Meropenem-resistant | 20 | 2−>128 | 8 | 128 |
| | Susceptible | 22 | 0.25-8 | 4 | 8 |
| Colistin | All | 77 | 0.06−>32 | 0.5 | 8 |
| | Amikacin-resistant | 20 | 0.25-2 | 0.5 | 1 |
| | Colistin-resistant | 15 | 4−>32 | 8 | >32 |
| | Meropenem-resistant | 20 | 0.25-1 | 0.5 | 1 |
| | Susceptible | 22 | 0.06-1 | 0.5 | 0.5 |
| Genta- | All | 77 | 0.06−>32 | 32 | >32 |
| micin | Amikacin-resistant | 20 | 8−>32 | >32 | >32 |
| | Colistin-resistant | 15 | 0.5−>32 | 32 | >32 |
| | Meropenem-resistant | 20 | 0.5−>32 | >32 | >32 |
| | Susceptible | 22 | 0.06-2 | 1 | 2 |
| Mero- | All | 77 | 0.06−>32 | 4 | >32 |
| penem | Amikacin-resistant | 20 | 0.5-32 | 2 | 16 |
| | Colistin-resistant | 15 | 0.06−>32 | 32 | >32 |
| | Meropenem-resistant | 20 | 16−>32 | >32 | >32 |
| | Susceptible | 22 | 0.06-4 | 0.25 | 2 |
| Tobra- | All | 77 | 0.12−>32 | 2 | >32 |
| mycin | Amikacin-resistant | 20 | 1−>32 | >32 | >32 |
| | Colistin-resistant | 15 | 0.5−>32 | 32 | >32 |
| | Meropenem-resistant | 20 | 0.5−>32 | 4 | >32 |
| | Susceptible | 22 | 0.12-2 | 1 | 2 |

*number of isolates

### Example 3

### Checkerboard Testing

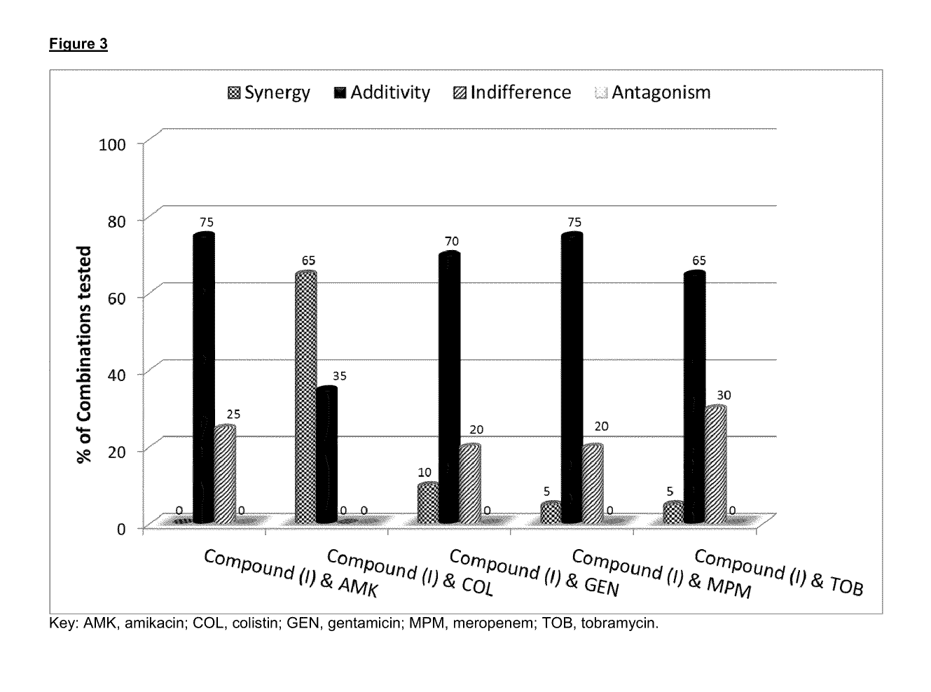

Checkerboard studies were carried out as described in General Procedures using twenty different A. baumannii isolates with 5 per sub-group of: susceptible, amikacin-resistant, colistin-resistant and meropenem-resistant. Combinations of compound (I) with amikacin, colistin, gentamicin, meropenem and tobramycin were tested against the different isolates and the results are set out below in Tables 2-5 and are summarized in FIG. 3.

TABLE 2
checkerboard interaction studies with compound
(I) and additional antibacterial agents against
susceptible isolates of A. baumannii

| Additional | | MIC add. | | Σ FIC | |
|---|---|---|---|---|---|
| antibacte- | MIC (I) | agent | Σ FIC | AVER- | |
| rial agent | (μg/ml) | (μg/ml) | RANGES | AGE | Conclusion |
| | A. baumannii 674340 | | | | |
| Amikacin | 2 | 4 | 0.50-4.06 | 1.56 | Indifference |
| Colistin | 1 | 0.5 | 0.38-1.06 | 0.64 | Additivity |
| Gentamicin | 2 | 1 | 0.50-1.13 | 0.80 | Additivity |
| Meropenem | 2 | 0.5 | 0.50-1.06 | 0.73 | Additivity |
| Tobramycin | 2 | 1 | 0.56-2.06 | 1.13 | Indifference |
| | A. baumannii 628760 | | | | |
| Amikacin | 2 | 8 | 0.56-1.25 | 0.94 | Additivity |
| Colistin | 2 | 0.5 | 0.25-1.03 | 0.52 | Additivity |
| Gentamicin | 2 | 1 | 0.56-1.13 | 0.91 | Additivity |
| Meropenem | 2 | 0.25 | 0.75-1.13 | 0.92 | Additivity |
| Tobramycin | 2 | 2 | 0.50-2.06 | 0.95 | Additivity |
| | A. baumannii 668116 | | | | |
| Amikacin | 4 | 2 | 0.31-1.06 | 0.71 | Additivity |
| Colistin | 1 | 1 | 0.25-0.53 | 0.36 | Synergy |
| Gentamicin | 1 | 0.5 | 0.75-1.25 | 1.04 | Indifference |
| Meropenem | 2 | 0.5 | 0.5-1.13 | 0.80 | Additivity |
| Tobramycin | 1 | 1 | 0.75-1.13 | 0.88 | Additivity |
| | A. baumannii 672190 | | | | |
| Amikacin | 8 | 4 | 0.38-1.13 | 0.77 | Additivity |
| Colistin | 4 | 0.5 | 0.25-0.53 | 0.36 | Synergy |
| Gentamicin | 4 | 1 | 0.53-1.25 | 0.97 | Additivity |
| Meropenem | 8 | 0.5 | 0.50-1.13 | 0.86 | Additivity |
| Tobramycin | 8 | 1 | 0.31-1.06 | 0.67 | Additivity |
| | A. baumannii 670348 | | | | |
| Amikacin | 2 | 8 | 0.63-1.25 | 1.02 | Indifference |
| Colistin | 2 | 0.5 | 0.50-0.63 | 0.58 | Additivity |
| Gentamicin | 2 | 1 | 0.63-2.13 | 1.52 | Indifference |
| Meropenem | 4 | 0.5 | 0.31-1.03 | 0.56 | Additivity |
| Tobramycin | 2 | 2 | 0.75-1.25 | 1.05 | Indifference |

(I) = compound (I);
add. agent = additional antibacterial agent

TABLE 3
checkerboard interaction studies with compound
(I) and additional antibacterial agents against
amikacin-resistant isolates of A. baumannii

| Additional | | MIC add. | | Σ FIC | |
|---|---|---|---|---|---|
| antibacte- | MIC (I) | agent | Σ FIC | AVER- | |
| rial agent | (μg/ml) | (μg/ml) | RANGES | AGE | Conclusion |
| | A. baumannii 637723 | | | | |
| Amikacin | 2 | 256 | 0.56-1.25 | 1.00 | Additivity |
| Colistin | 2 | 0.5 | 0.38-0.56 | 0.47 | Synergy |
| Gentamicin | 2 | 128 | 0.50-1.13 | 0.80 | Additivity |
| Meropenem | 2 | 1 | 0.50-0.63 | 0.58 | Additivity |
| Tobramycin | 2 | 2 | 0.53-1.13 | 0.87 | Additivity |
| | A. baumannii 638115 | | | | |
| Amikacin | 4 | 1024 | 0.38-1.13 | 0.77 | Additivity |
| Colistin | 4 | 0.5 | 0.25-1.06 | 0.69 | Additivity |
| Gentamicin | 4 | 4 | 0.50-1.06 | 0.68 | Additivity |
| Meropenem | 8 | 2 | 0.50-1.06 | 0.75 | Additivity |
| Tobramycin | 4 | 2 | 0.63-1.06 | 0.81 | Additivity |
| | A. baumannii 661954 | | | | |
| Amikacin | 4 | 512 | 0.56-1.00 | 0.73 | Additivity |
| Colistin | 4 | 1 | 0.19-0.53 | 0.32 | Synergy |
| Gentamicin | 4 | 16 | 1.06-1.25 | 1.15 | Indifference |
| Meropenem | 8 | 1 | 0.75-1.25 | 1.04 | Indifference |
| Tobramycin | 4 | 1 | 0.75-1.25 | 1.05 | Indifference |
| | A. baumannii 668239 | | | | |
| Amikacin | 4 | >1024 | 0.38-1.06 | 0.64 | Additivity |
| Colistin | 2 | 0.5 | 0.38-0.75 | 0.54 | Additivity |
| Gentamicin | 2 | 1024 | 0.38-0.63 | 0.51 | Additivity |
| Meropenem | 4 | 16 | 0.75-1.13 | 0.92 | Additivity |
| Tobramycin | 8 | 4 | 0.38-1.06 | 0.68 | Additivity |
| | A. baumannii 655932 | | | | |
| Amikacin | 4 | 512 | 0.28-1.06 | 0.65 | Additivity |
| Colistin | 2 | 0.5 | 0.31-0.63 | 0.45 | Synergy |
| Gentamicin | 2 | 128 | 0.25-0.56 | 0.40 | Synergy |
| Meropenem | 4 | 16 | 0.16-0.53 | 0.27 | Synergy |
| Tobramycin | 2 | 2 | 0.50-1.13 | 0.81 | Additivity |

(I) = compound (I);
add. agent = additional antibacterial agent

TABLE 4
checkerboard interaction studies with compound
(I) and additional antibacterial agents against
colistin-resistant isolates of A. baumannii

| Additional | | MIC add. | | Σ FIC | |
|---|---|---|---|---|---|
| antibacte- | MIC (I) | agent | Σ FIC | AVER- | |
| rial agent | (μg/ml) | (μg/ml) | RANGES | AGE | Conclusion |
| | A. baumannii 441142 | | | | |
| Amikacin | 4 | >1024 | 0.56-1.13 | 0.81 | Additivity |
| Colistin | 2 | 8 | 0.38-0.63 | 0.5 | Synergy |
| Gentamicin | 2 | >1024 | 0.50-1.25 | 0.96 | Additivity |
| Meropenem | 4 | 64 | 1.00-1.25 | 1.13 | Indifference |
| Tobramycin | 4 | >1024 | 1.00-1.25 | 1.13 | Indifference |
| | A. baumannii 441160 | | | | |
| Amikacin | 8 | >1024 | 0.63-1.25 | 1.02 | Indifference |
| Colistin | 4 | 4 | 0.28-1.13 | 0.65 | Additivity |
| Gentamicin | 8 | >1024 | 0.53-1.13 | 0.87 | Additivity |
| Meropenem | 8 | 64 | 0.63-1.06 | 0.80 | Additivity |
| Tobramycin | 4 | >1024 | 1.13-1.50 | 1.29 | Indifference |
| | A. baumannii 463996 | | | | |
| Amikacin | 4 | 8 | 0.28-1.13 | 0.74 | Additivity |
| Colistin | 2 | 4 | 0.38-0.56 | 0.44 | Synergy |
| Gentamicin | 4 | 2 | 0.25-0.56 | 0.36 | Synergy |
| Meropenem | 4 | 0.125 | 0.75-2.13 | 1.55 | Indifference |
| Tobramycin | 4 | 2 | 0.31-1.06 | 0.59 | Additivity |
| | A. baumannii 466936 | | | | |
| Amikacin | 8 | 64 | 0.38-2.13 | 1.19 | Indifference |
| Colistin | 8 | 8 | 0.19-0.56 | 0.38 | Synergy |
| Gentamicin | 16 | 4 | 0.31-1.06 | 0.70 | Additivity |
| Meropenem | 8 | 128 | 0.75-1.13 | 0.98 | Additivity |
| Tobramycin | 8 | 64 | 0.38-1.13 | 0.80 | Additivity |
| | A. baumannii 517303 | | | | |
| Amikacin | 4 | 128 | 0.38-1.06 | 0.64 | Additivity |
| Colistin | 4 | >1024 | 0.09-0.50 | 0.24 | Synergy |
| Gentamicin | 4 | 512 | 0.25-1.06 | 0.53 | Additivity |
| Meropenem | 4 | 1 | 0.52-1.25 | 0.99 | Additivity |
| Tobramycin | 4 | 256 | 0.27-0.63 | 0.49 | Synergy |

(I) = compound (I);
add. agent = additional antibacterial agent

TABLE 5
checkerboard interaction studies with compound
(I) and additional antibacterial agents against
meropenem-resistant isolates of A. baumannii

| Additional | | MIC add. | | Σ FIC | |
|---|---|---|---|---|---|
| antibacte- | MIC (I) | agent | Σ FIC | AVER- | |
| rial agent | (μg/ml) | (μg/ml) | RANGES | AGE | Conclusion |
| | A. baumannii 618649 | | | | |
| Amikacin | 2 | 4 | 0.63-0.75 | 0.71 | Additivity |
| Colistin | 4 | 0.5 | 0.31-0.38 | 0.35 | Synergy |
| Gentamicin | 4 | 0.5 | 0.75-2.06 | 1.31 | Indifference |
| Meropenem | 4 | 64 | 0.75-2.06 | 1.23 | Indifference |
| Tobramycin | 4 | 1 | 0.53-1.06 | 0.74 | Additivity |
| | A. baumannii 625917 | | | | |
| Amikacin | 4 | 4 | 0.53-2.13 | 1.37 | Indifference |
| Colistin | 4 | 0.5 | 0.25-0.31 | 0.29 | Synergy |
| Gentamicin | 4 | 1 | 0.75-1.13 | 0.98 | Additivity |
| Meropenem | 4 | 64 | 0.63-1.13 | 0.89 | Additivity |
| Tobramycin | 4 | 1 | 0.63-2.13 | 1.39 | Indifference |
| | A. baumannii 650216 | | | | |
| Amikacin | 8 | 4 | 0.31-1.13 | 0.83 | Additivity |
| Colistin | 8 | 0.25 | 0.37-0.56 | 0.47 | Synergy |
| Gentamicin | 8 | 64 | 0.50-0.63 | 0.56 | Additivity |
| Meropenem | 8 | 64 | 0.50-1.06 | 0.69 | Additivity |
| Tobramycin | 8 | 1 | 0.75-1.13 | 0.98 | Additivity |
| | A. baumannii 671050 | | | | |
| Amikacin | 4 | 8 | 0.063-1.06 | 0.86 | Additivity |
| Colistin | 4 | 0.5 | 0.38-0.63 | 0.52 | Additivity |
| Gentamicin | 4 | 8 | 0.75-1.13 | 0.88 | Additivity |
| Meropenem | 8 | 32 | 0.63-1.13 | 0.89 | Additivity |
| Tobramycin | 4 | 4 | 0.75-1.13 | 0.92 | Additivity |
| | A. baumannii 667848 | | | | |
| Amikacin | 4 | 4 | 0.50-0.63 | 0.55 | Additivity |
| Colistin | 8 | 0.5 | 0.25-0.56 | 0.38 | Synergy |
| Gentamicin | 4 | 256 | 0.38-1.13 | 0.67 | Additivity |
| Meropenem | 8 | 32 | 0.38-0.63 | 0.52 | Additivity |
| Tobramycin | 4 | 1 | 0.50-1.13 | 0.81 | Additivity |

(I) = compound (I);
add. agent = additional antibacterial agent

### CONCLUSION

None of the combinations of compound (I)/additional antibacterial agent tested exhibited antagonism and only 19% exhibited indifference. Irrespective of the resistance phenotypes, compound (I)/additional antibacterial agent combinations assessed resulted in synergy or additivity in 81% of combinations (additivity in 64% of combinations and synergy in 17% of combinations). The best effect was shown with compound (I) combined with colistin where 65% of the combinations were synergistic.

### Example 4

### Time Kill Kinetics—Bactericidal Effects Against Various A. baumannii Isolates

Time-kill studies were carried out as described in General Procedures using combinations of compound (I) with amikacin, meropenem and colistin, respectively.

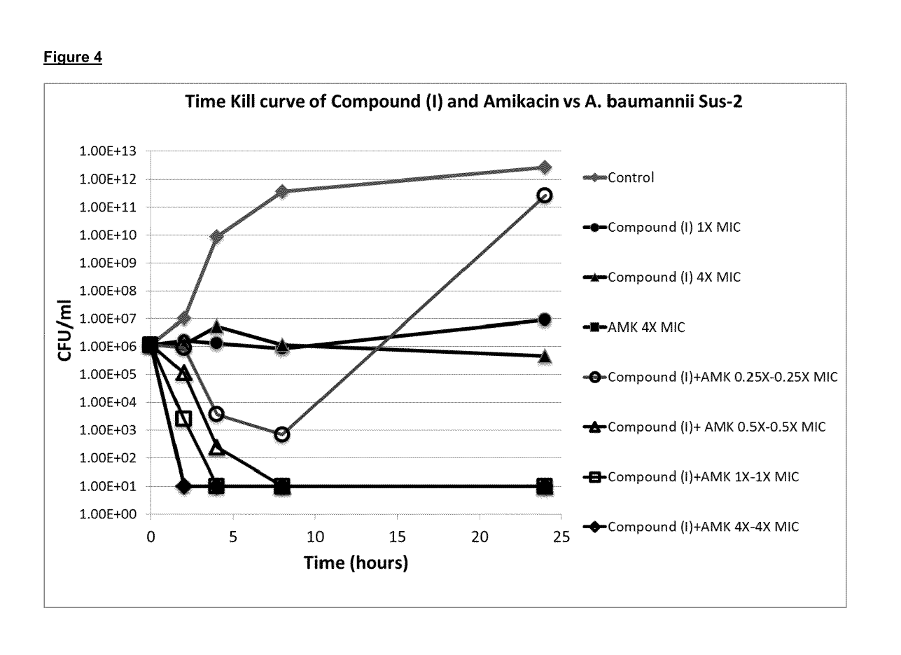

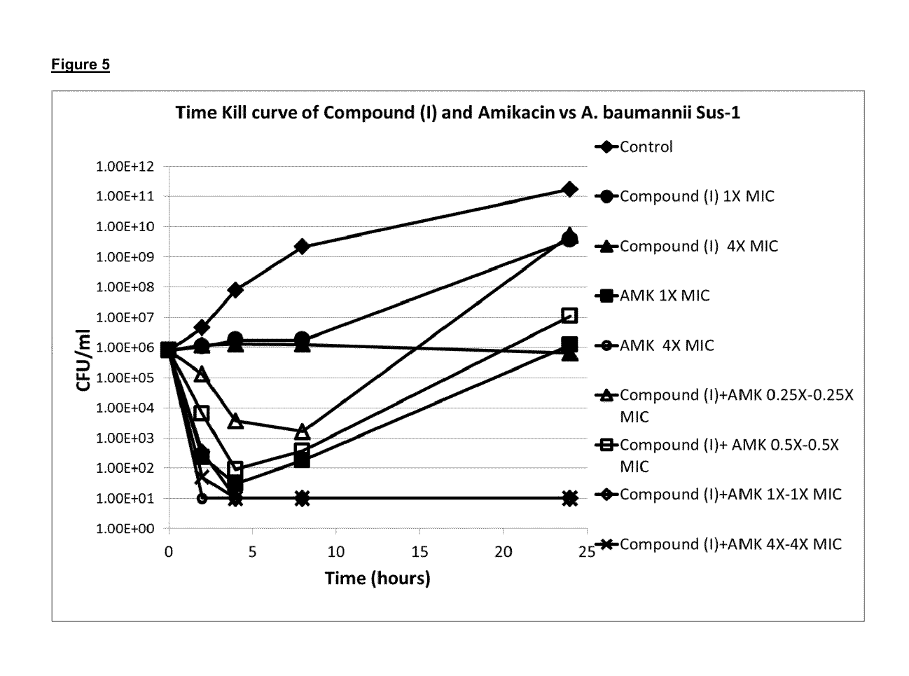

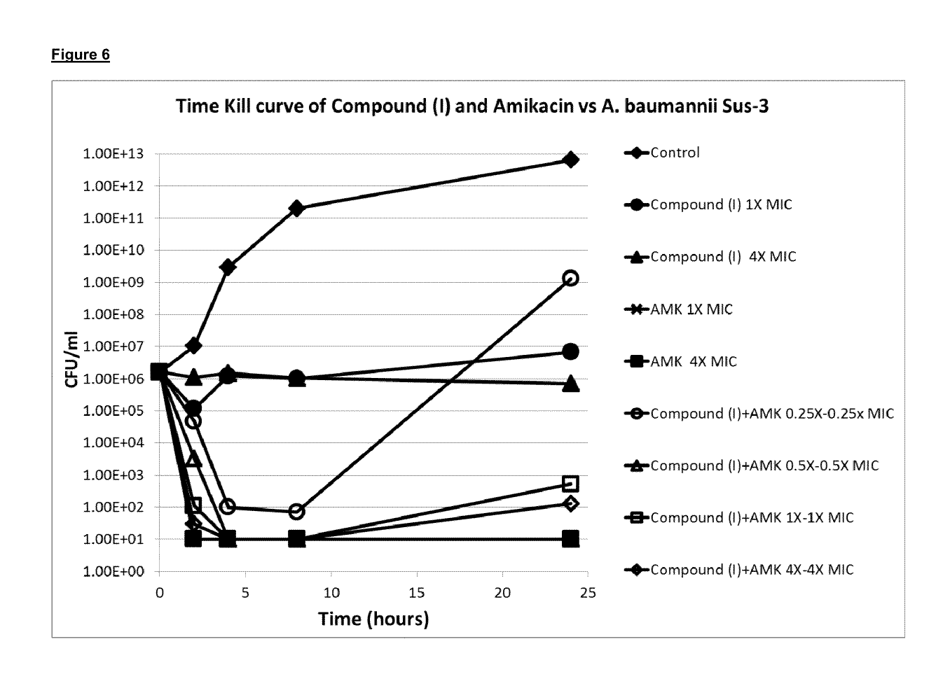

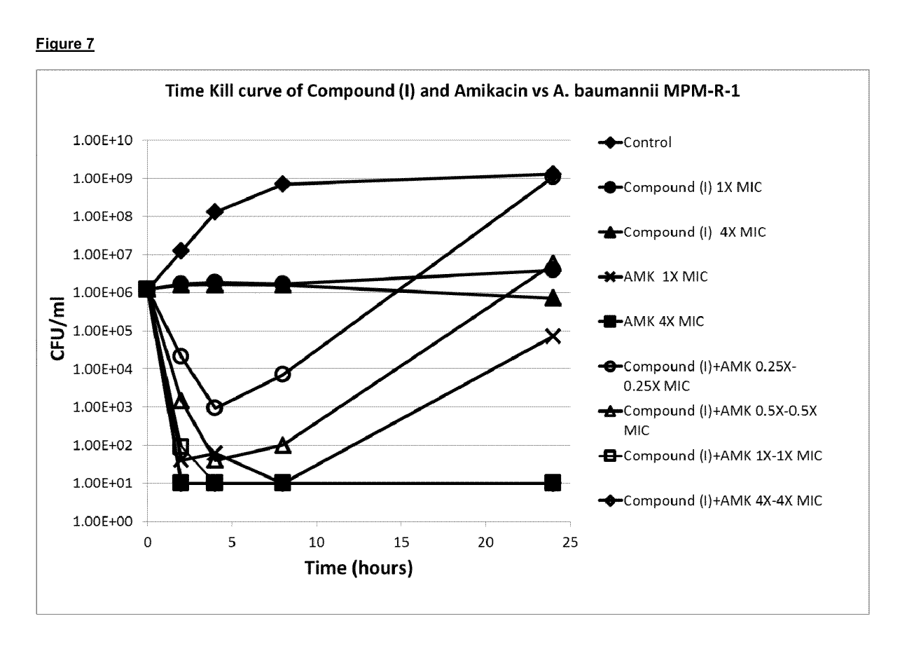

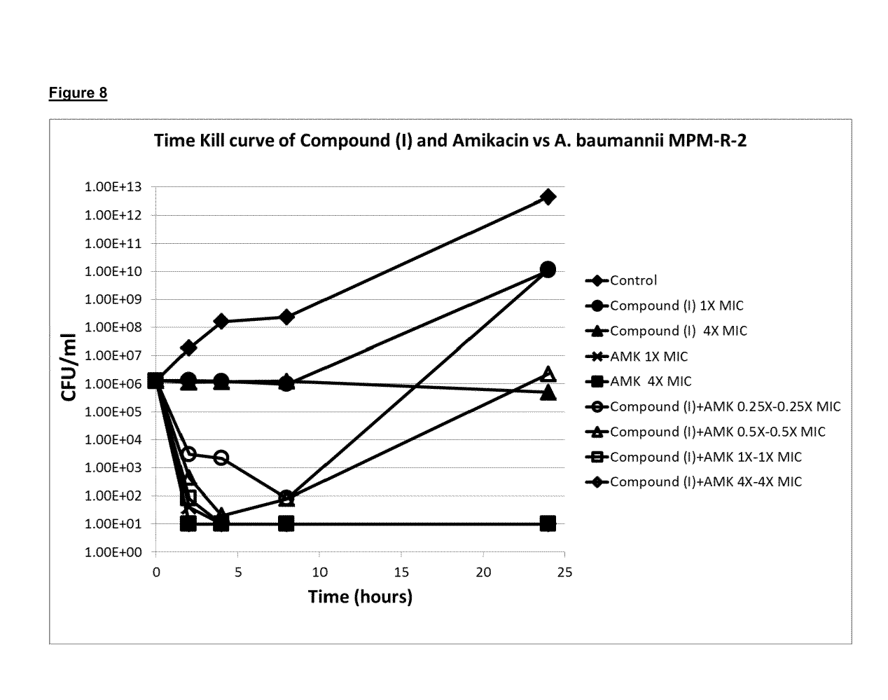

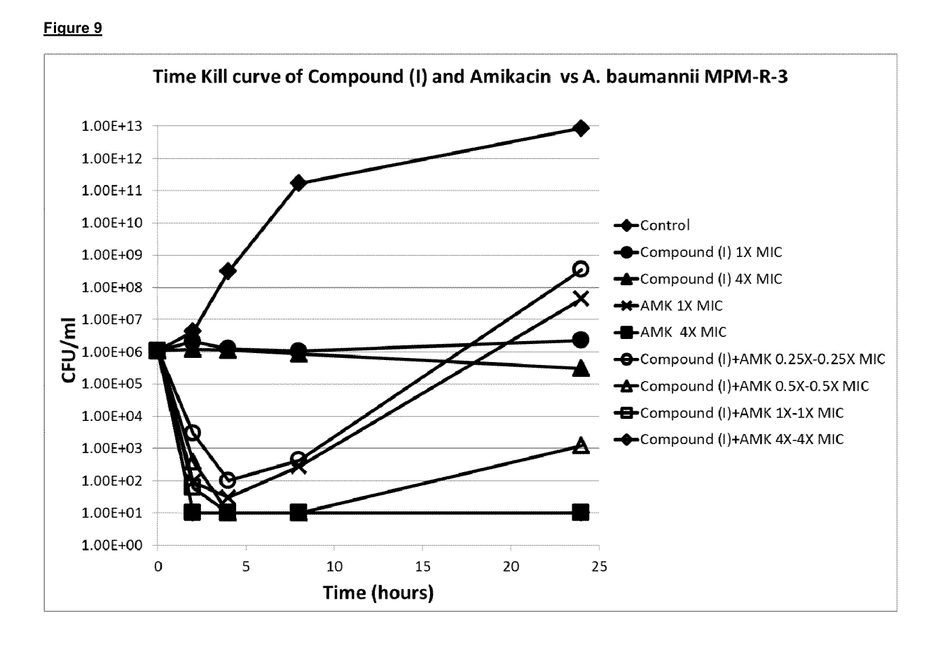

The bactericidal effects of compound (I) in combination with amikacin (AMK) against various A. baumannii isolates (phenotypes) are illustrated in FIGS. 4-9 and summarised in Table 6.

TABLE 6

| | | | Time to kill 99.9% | | | | | | | |
|---|---|---|---|---|---|---|---|---|---|---|
| | | | Δ3log₁₀ reduction compared to initial inoculum | | | | | | | |
| | | | | | | | IN COMBINATION | | | |
| | | | | | | | AMK/ | AMK/ | | |
| | | | ALONE | | | | (1) | (1) | AMK/ | AMK/ |
| | MIC (μg/mL) | | (1) | (1) | AMK | AMK | 0.25x/ | 0.5x/ | (1) | (1) |
| Phenotype | (1) | AMK | 1xMIC | 4xMIC | 1xMIC | 4xMIC | 0.25x | 0.5x | 1x/1x | 4x/4x |
| Sus-1 | 1 | 2 | >24 h | >24 h | 1.7 h* | 1.2 h | >24 h | 3 h* | 1.8 h | 1.4 h |
| Sus-2 | 2 | 4 | >24 h | >24 h | ND | 1.2 h | 6.8 h* | 3.5 h | 2.3 h | 1.2 h |
| Sus-3 | 8 | 4 | >24 h | >24 h | 1.3 h | 1.3 h | 3.1 h* | 2.2 h | 1.4 h | 1.3 h |
| MPM-R-1 | 8 | 4 | >24 h | >24 h | 1.4 h* | 1.2 h | 3.8 h* | 2.1 h* | 1.5 h | 1.2 h |
| MPM-R-2 | 4 | 8 | >24 h | >24 h | 1.3 h | 1.2 h | 4.7 h* | 1.8 h* | 1.4 h | 1.2 h |
| MPM-R-3 | 4 | 4 | >24 h | >24 h | 1.4 h* | 1.2 h | 2.6 h* | 1.8 h | 1.4 h | 1.2 h |

*Δ3Log₁₀ kill observed during 24 hour experimental period though re-growth also observed.
Sub-MIC combinations that resulted in Δ3Log₁₀ kill
Sus = susceptible;
MPM-R = meropenem-resistant
(1) = Compound (I);
AMK = Amikacin
ND, not determined

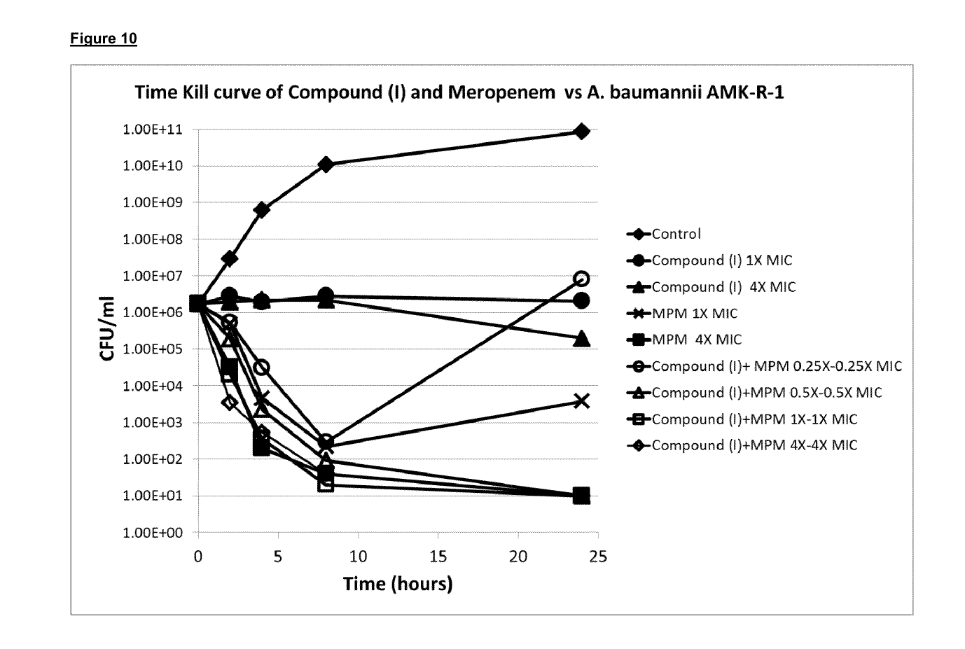

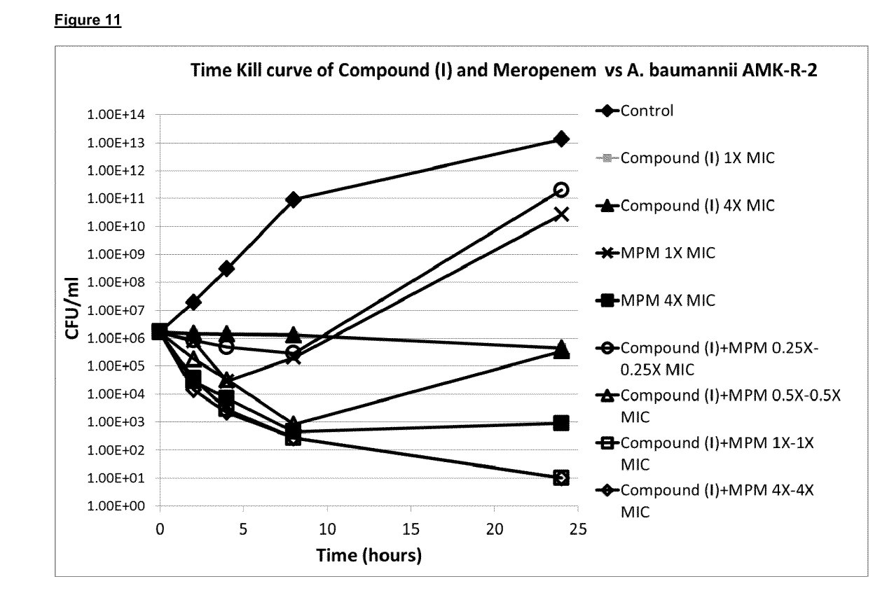

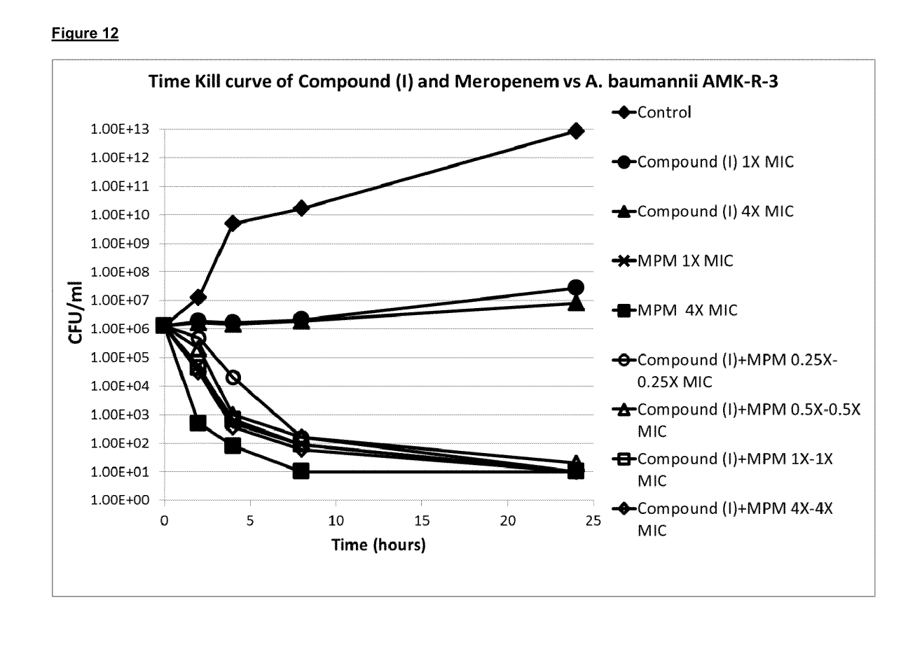

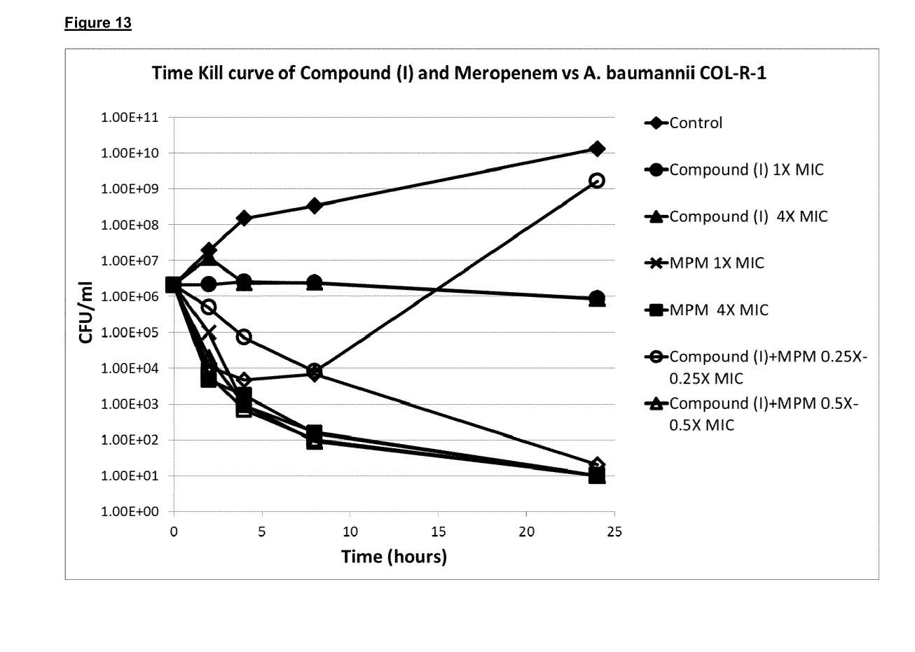

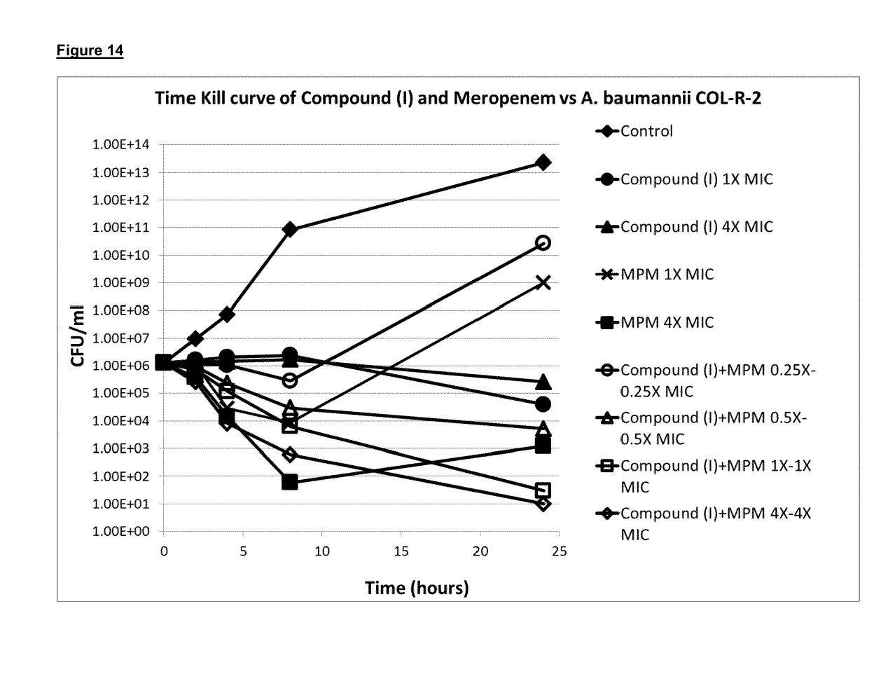

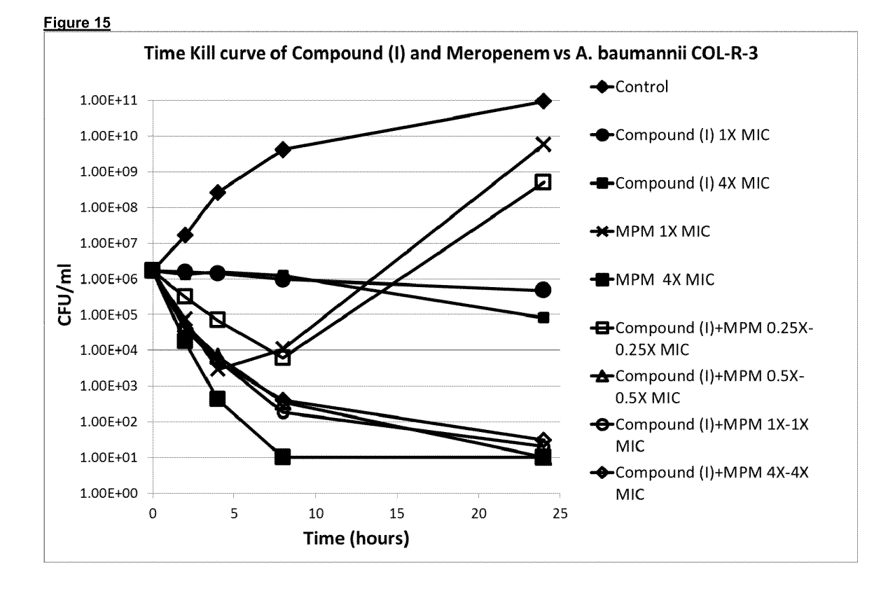

The bactericidal effects of compound (I) in combination with meropenem (MPM) against various A. baumannii isolates (phenotypes) are illustrated in FIGS. 10-15 and summarised in Table 7.

TABLE 7

| | | | Time to kill 99.9% | | | | | | | |
|---|---|---|---|---|---|---|---|---|---|---|
| | | | Δ3log₁₀ reduction compared to initial inoculum | | | | | | | |
| | | | | | | | IN COMBINATION | | | |
| | | | | | | | MPM/ | MPM/ | | |
| | | | ALONE | | | | (1) | (1) | MPM/ | MPM/ |
| | MIC (μg/mL) | | (1) | (1) | MPM | MPM | 0.25x/ | 0.5x/ | (1) | (1) |
| Phenotype | (1) | MPM | 1xMIC | 4xMIC | 1xMIC | 4xMIC | 0.25x | 0.5x | 1x/1x | 4x/4x |
| AMK-R-1 | 4 | 4 | >24 h | >24 h | 5.2 h* | 3.2 | 6.5 h* | 4.3 h | 3.2 h | 2.7 h |
| AMK-R-2 | 4 | 16 | >24 h | >24 h | >24 h | 6.1 h | >24 h | 7.2 h* | 4.9 h | 4.4 h |
| AMK-R-3 | 2 | 16 | >24 h | >24 h | 3.6 h | 1.8 h | 6.3 h | 3.9 h | 3.7 h | 3.5 h |
| COL-R-1 | 8 | 64 | >24 h | >24 h | 3.6 h | 3.6 h | >24 h | 3.5 h | 3.0 h | 11.3 h |
| COL-R-2 | 4 | 0.25 | >24 h | >24 h | >24 h | 5.8 h* | >24 h | >24 h | 12.8 h | 6.8 h |
| COL-R-3 | 16 | 16 | >24 h | >24 h | >24 h | 3.2 h | >24 h | 5.9 h | 5.4 h | 5.6 h |

*Δ3Log₁₀ kill observed during 24 hour experimental period though re-growth also observed
Sub-MIC combinations that resulted in Δ3Log₁₀ kill
AMK-R = amikacin-resistant;
COL-R = colistin-resistant
(1) = Compound (I);
MPM = meropenem

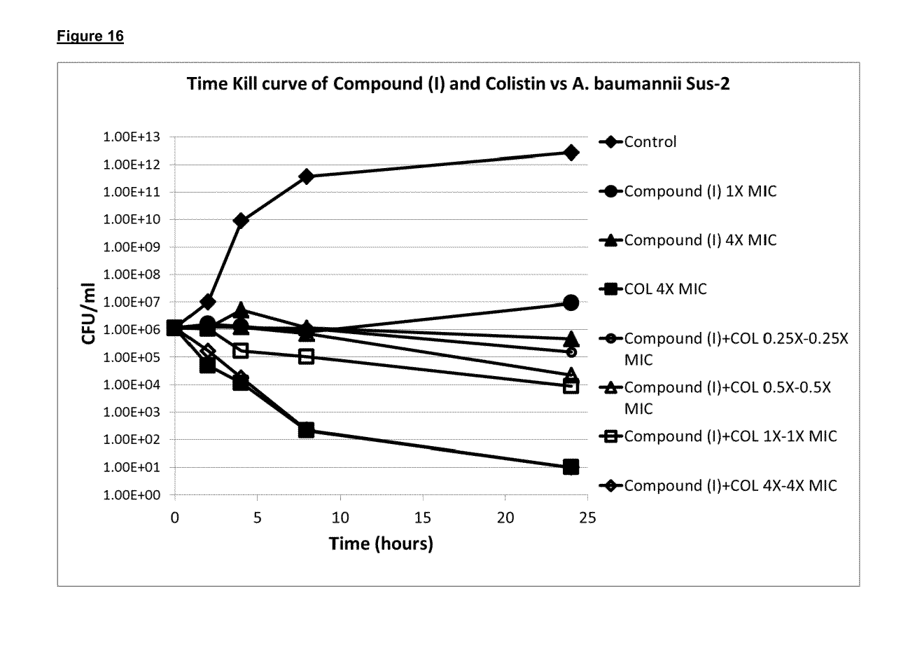

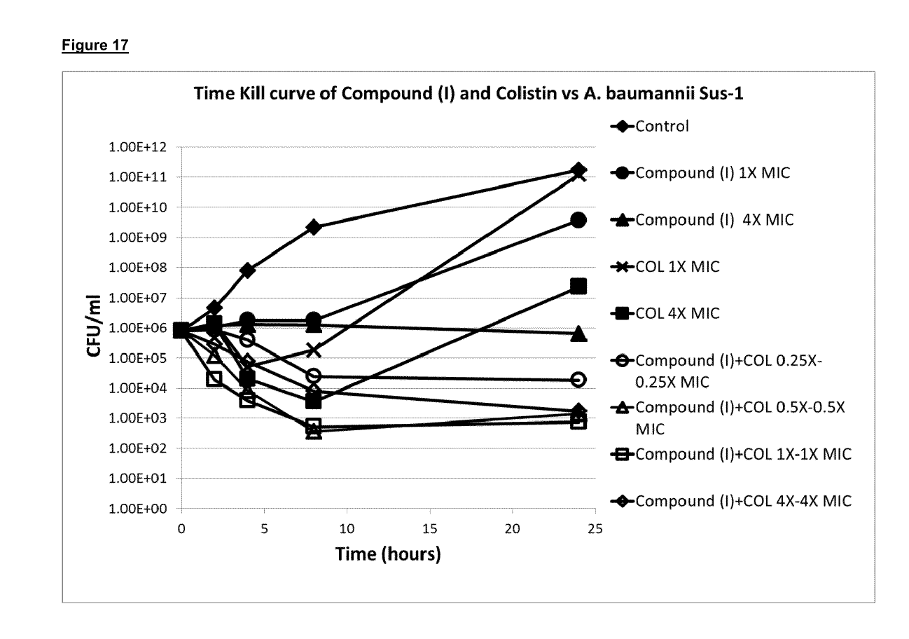

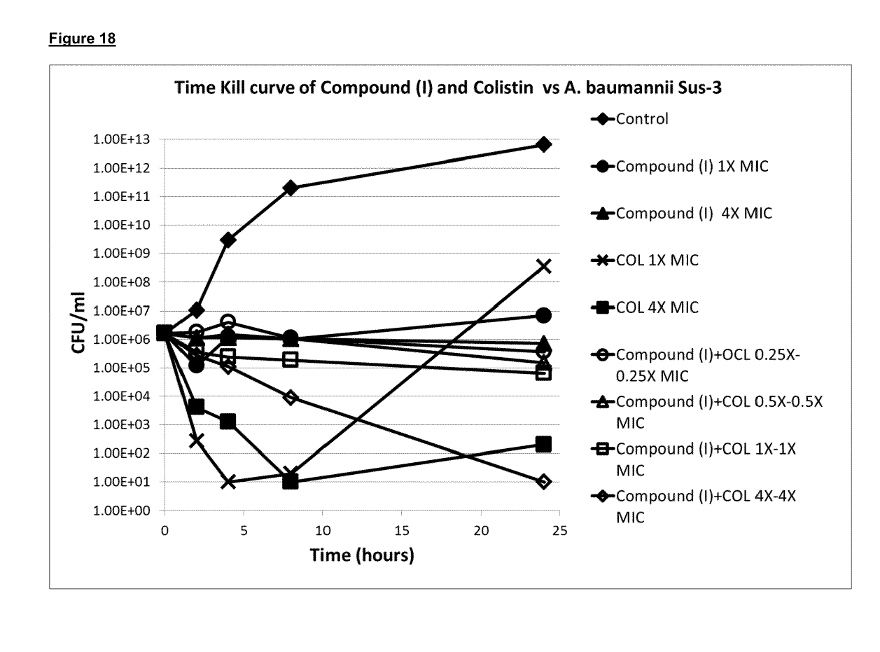

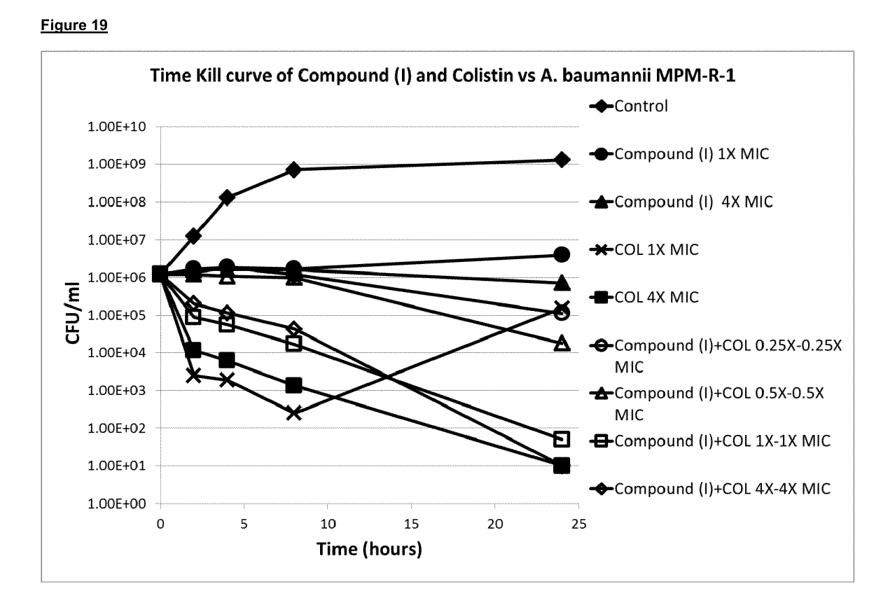

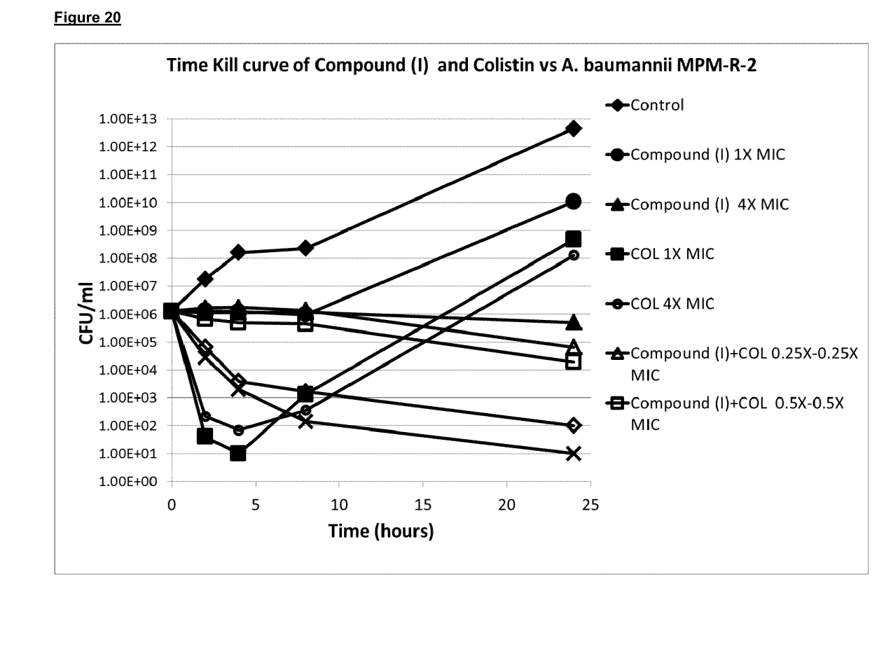

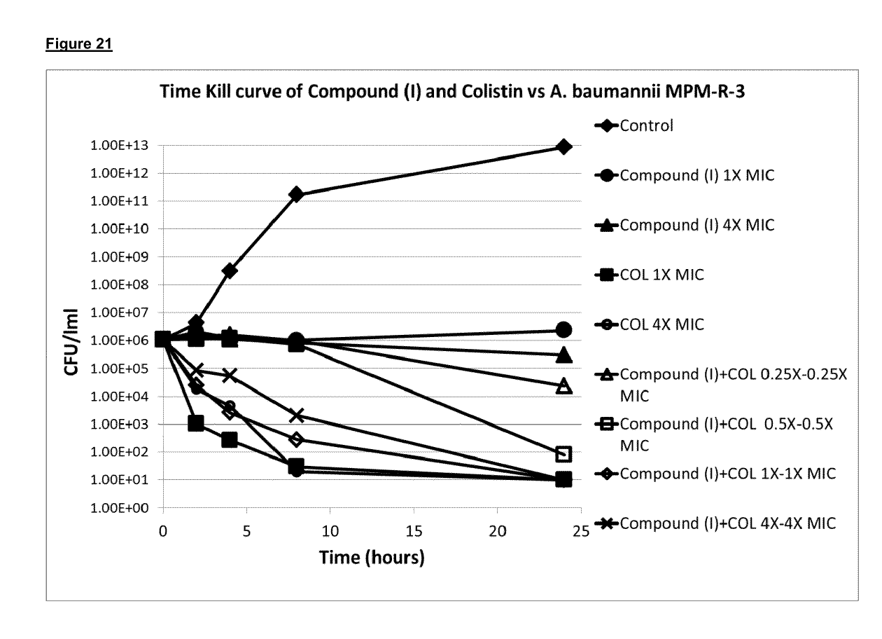

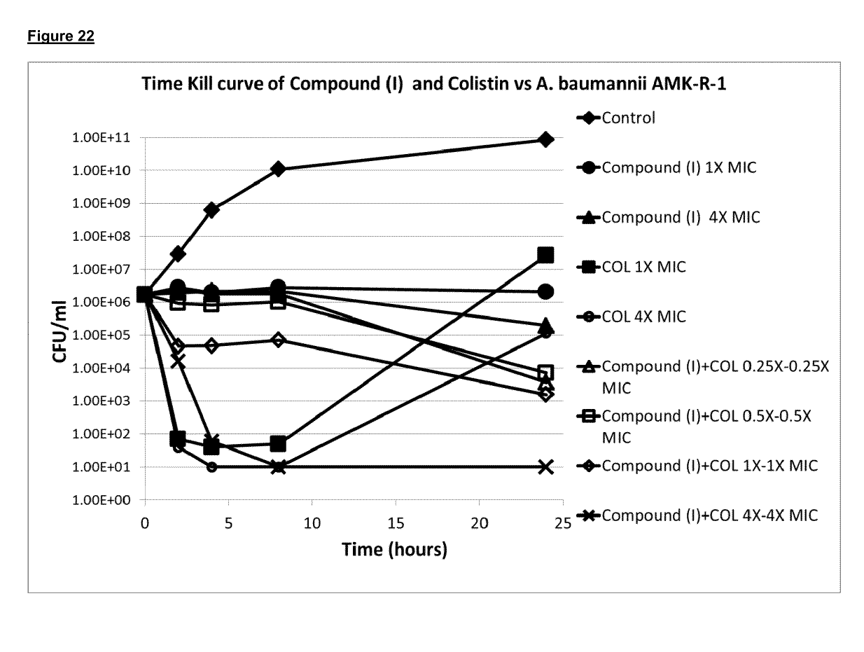

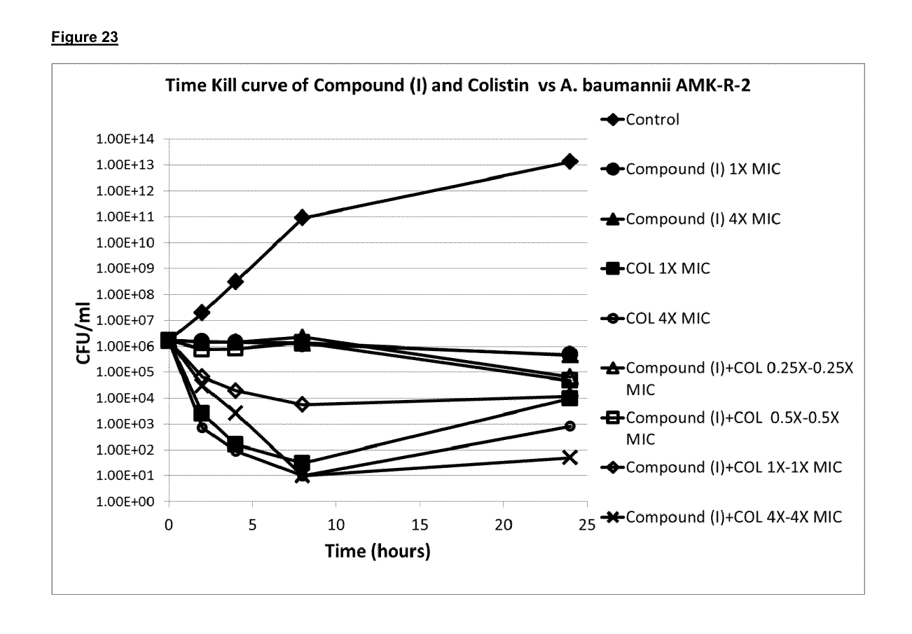

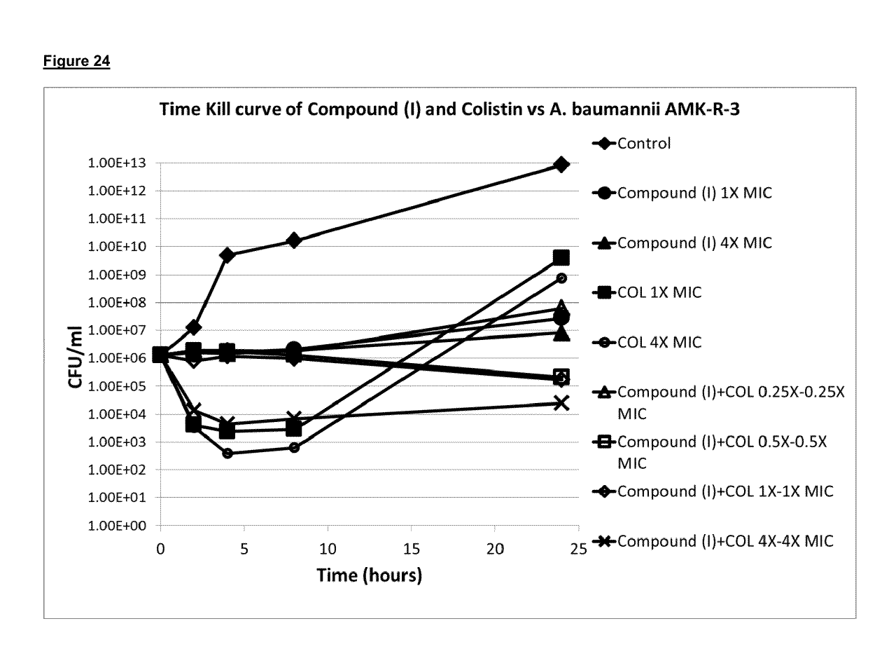

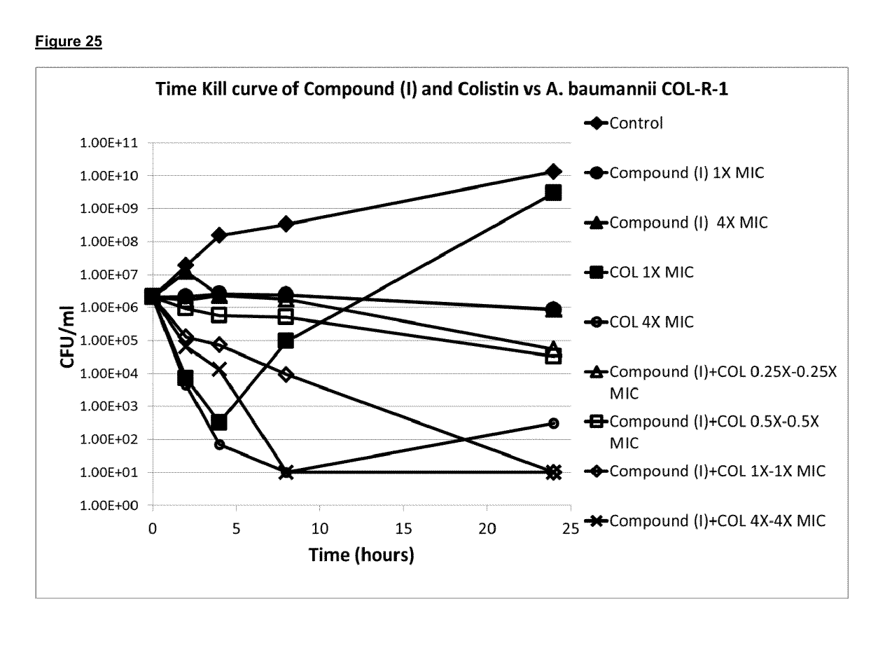

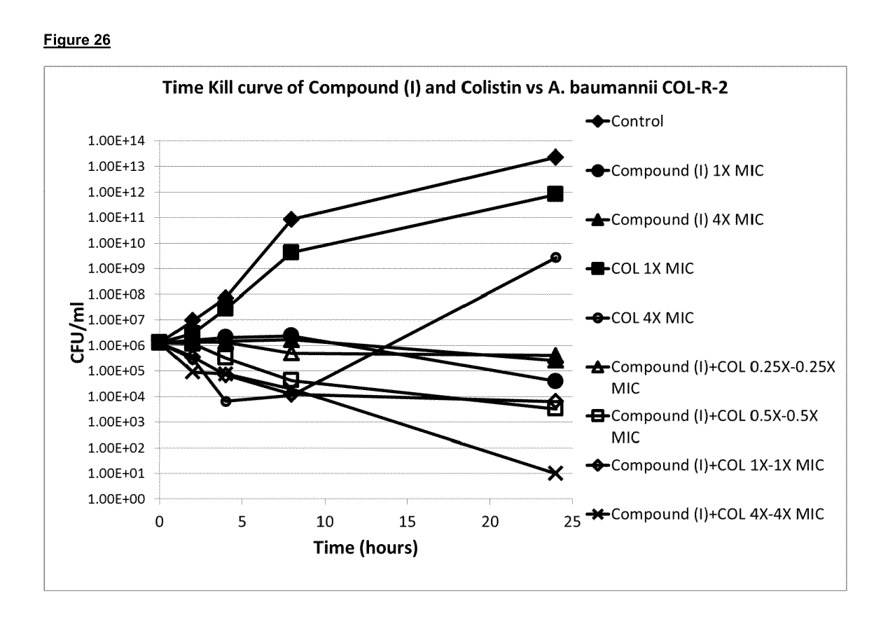

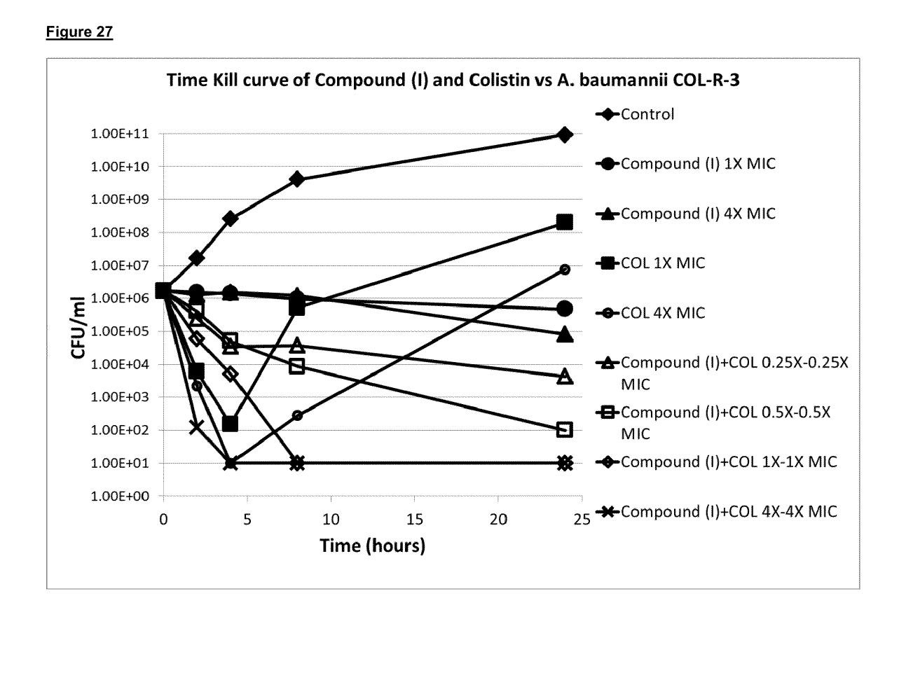

The bactericidal effects of compound (I) in combination with colistin (COL) against various A. baumannii isolates (phenotypes) are illustrated in FIGS. 16-27 and summarised in Table 8.

TABLE 8

| | | | Time to kill 99.9% | | | | | | | |
|---|---|---|---|---|---|---|---|---|---|---|
| | | | Δ3log₁₀ reduction compared to initial inoculum | | | | | | | |
| | | | | | | | IN COMBINATION | | | |
| | | | | | | | COL/ | COL/ | | |
| | | | ALONE | | | | (1) | (1) | COL/ | COL/ |
| | MIC (μg/mL) | | (1) | (1) | COL | COL | 0.25x/ | 0.5x/ | (1) | (1) |
| Phenotype | (1) | COL | 1xMIC | 4xMIC | 1xMIC | 4xMIC | 0.25x | 0.5x | 1x/1x | 4x/4x |
| Sus-2 | 2 | 0.5 | >24 h | >24 h | | 6.3 h | >24 h | >24 h | >24 h | 6.5 h |
| Sus-1 | 1 | 1 | >24 h | >24 h | >24 h | >24 h | >24 h | 7.0 h* | 7.2 h | >24 h |
| Sus-3 | 8 | 0.25 | >24 h | >24 h | 1.6 h* | 3.6 h | >24 h | >24 h | >24 h | 12.0 h |
| MPM-R-1 | 8 | 0.25 | >24 h | >24 h | 5.0 h* | 8.1 h | >24 h | >24 h | 15.1 h | 14.7 h |
| MPM-R-1 | 4 | 1 | >24 h | >24 h | 1.3 h* | 1.6 h* | >24 h | >24 h | 9.6 h | 4.7 h |
| MPM-R-3 | 4 | 1 | >24 h | >24 h | 2 h | 5.1 h | >24 h | 19.4 h | 5.6 h | 9.8 h |
| AMK-R-1 | 4 | 0.5 | >24 h | >24 h | 1.4 h* | 1.3 h* | >24 h | >24 h | 23.5 h | 2.8 h |
| AMK-R-2 | 4 | 0.5 | >24 h | >24 h | 2.3 h* | 1.8 h | >24 h | >24 h | >24 h | 4.3 h |
| AMK-R-3 | 2 | 0.25 | >24 h | >24 h | >24 h | 2.9 h* | >24 h | >24 h | >24 h | >24 h |
| COL-R-1 | 8 | 8 | >24 h | >24 h | 2.8 h* | 2.3 h | >24 h | >24 h | 11.4 h | 5.0 h |
| COL-R-2 | 4 | 4 | >24 h | >24 h | >24 h | >24 h | >24 h | >24 h | >24 h | 13.7 h |
| COL-R-3 | 16 | 16 | >24 h | >24 h | 2.7 h* | 2.1 h* | >24 h | 13.9 h | 4.7 h | 1.5 h |

*Δ3Log₁₀ kill observed during 24 hour experimental period though re-growth also observed.
Sub-MIC combinations that resulted in Δ3Log₁₀ kill.
Sus = susceptible;
MPM-R = meropenem-resistant;
COL-R = colistin-resistant
(1) = Compound (I);
COL = colistin

In time-kill studies, compound (I) when tested alone at either 1× or 4×MIC was bacteriostatic.

The additional antibacterial agents, amikacin, meropenem and colistin, were generally bactericidal (99.9% bacterial killing) against the majority of isolates when tested at 4×MIC and against many isolates when tested at 1×MIC.

In general, compound (I) 4×/additional antibacterial agent 4×MIC combinations resulted in the same effects as when the comparator was tested alone. This also held true for compound (I) 1×/additional antibacterial agent 1×MIC combinations though not for all isolates tested.

A most interesting finding was observed for sub-MIC combinations (0.25×/0.25×MIC and 0.5×/0.5×MIC) of compound (I)/amikacin or meropenem, whereby against the majority of the isolates tested, these sub-MIC concentrations tested exhibited bactericidal (time to attain 99.9% bacterial killing) activity (91.6% of amikacin experiments and 58.3% of meropenem experiments).

The combination of compound (I)/colistin at sub-MIC was bactericidal in 12.5% of experiments in this study.

### Conclusion—In Vitro Studies of Examples 2-4

In a study of activity in a panel of 77 isolates of A. baumannii, compound (I) exhibited good activity against the majority of the 77 isolates tested. Although the overall MIC range was quite broad, the majority of MICs were in the range of 2-8 μg/ml.

Checkerboard studies resulted in 81% of combinations demonstrating additivity or synergy, with combinations of compound (I) and gentamicin, meropenem and tobramycin demonstrating synergy against certain amikacin-resistant and colistin-resistant strains of A. baumannii and the combination of compound (I) with colistin resulted in 65% synergistic activity, against many susceptible, amikacin-resistant, colistin-resistant and meropenem-resistant strains of A. baumannii.

In a study of in vitro time kill in a panel of 20 isolates of A. baumannii, compound (I) when tested alone at 1× and 4×MIC was bacteriostatic, but sub-MIC combinations of compound (I)/amikacin or compound (I)/meropenem (0.25×/0.25×MIC and 0.5×/0.5×MIC) exhibited bactericidal activity against the majority of the 20 isolates tested. Despite the strong synergy demonstrated with the combination of compound (I) and colistin in the checkerboard experiments, a weaker interaction was observed in this particular study.

### Example 5

### Further In Vitro Study

Following on from the promising results of the in vitro experiments of Examples 2, 3 and 4, a smaller-scale in vitro study was carried out using the A. baumannii strains used in the in vivo mouse model study (Example 6).

### Materials

### Antimicrobial Agents

Compound (I) was supplied by NOVASEP-FINORGA. Methanol was added to 5 mg of compound (I) in order to obtain a 20 mg/mL solution.

Amikacin was supplied by MYLAN LAB. A solution containing 50 mg/mL of amikacin was diluted in saline solution and used immediately after dilution in order to obtain a concentration of 1.8 mg/mL.

Meropenem was supplied by ASTRA ZENECA as Meronem®. A solution containing 50 mg/mL of meropenem was diluted in saline solution and used immediately after dilution in order to obtain a concentration of 5 mg/mL.

Colistin was supplied by SANOFI-AVENTIS as Colimycine®. 3 mL of saline solution was added onto 33.33 mg of colistin powder in order to obtain a 11.11 mg/mL solution.

A. baumanni Isolates

A. baumannii CIP 5377 (ATCC® 17978): low cephalosporinase producing strain, susceptible to meropenem and amikacin, isolated in an infected patient

A. baumannii CIP 7034: low cephalosporinase producing strain, susceptible to meropenem and amikacin A. baumannii AYE ATCC® BAA1710™: extended spectrum beta-lactamase producing strain susceptible to meropenem and resistant to amikacin

A. baumannii SAN-94040 (SAN): cephalosporinase-overproducing strain, susceptible to meropenem and intermediate to amikacin and resistant to fluoroquinolones, isolated from blood cultures of an intensive care patient with nosocomial pneumonia from North Africa (Algeria).

A. baumannii RCH-69: cephalosporinase producing strain; low susceptibility to imipenem and resistant to amikacin.

Antimicrobial Susceptibility Testing (MIC Determination)

Minimum inhibitory concentration (MIC) endpoints were determined by broth microdilution methodology in a 96-well microplate after incubation for 24 hours at 37° C. according to the recommendations of EUCAST. The MICs results are given in Table 9:

TABLE 9
MIC data of compound (I) and comparator antibacterial agents

| MIC (μg/mL) | CIP 5377 | CIP 7034 | AYE | SAN | RCH |
|---|---|---|---|---|---|
| amikacin | 2 | 8 | 32 | 4 | >32 |
| meropenem | 0.25 | 1 | 1 | 1 | 32 |
| colistin | 0.5 | 0.5 | 1 | 1 | 0.5 |
| Compound (I) | 2 | 1 | 2 | 2 | 2 |

Compound (I) exhibited good activity against the 5 isolates of A. baumannii tested in this study with MICs ranging from 1 to 2 μg/mL against susceptible, amikacin-R and meropenem-R isolates.

Bactericidal Activity (Time Kill Kinetics)

Time-kill kinetics were performed at 37° C. by testing compound (I) and selected antimicrobial agents individually at 2× the corresponding MIC of each antibacterial agent. In addition, a second set of experiments involved the testing of compound(I)/additional antibacterial agent combinations of 2×/2×MIC. Samples were taken at 0, 3, 6 and 24 hours in order to determine colony forming units (CFU/mL). Bactericidal activity was defined by a reduction of the inoculum by 3 log 10 compared to initial inoculum (10⁵ CFU/mL) at a defined time point (CLSI M26A, 1999).

In this time-kill study, compound (I) was observed to be bacteriostatic when tested alone at 2×MIC. The additional antibacterial agents amikacin and meropenem were observed to be bactericidal when tested alone at 2×MIC.

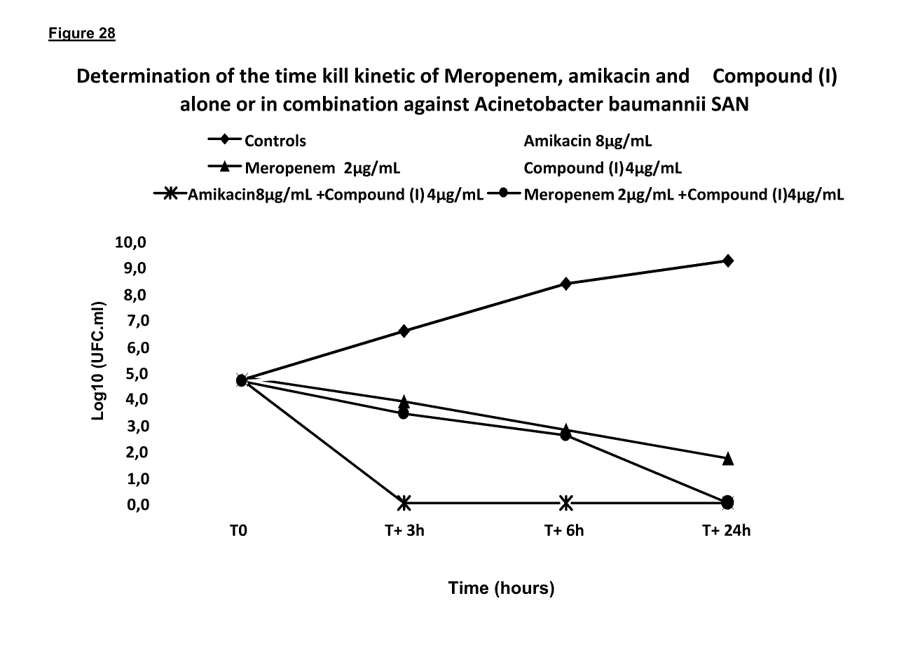

Combinations of compound (I) and either amikacin or meropenem (with all antibacterial agents at 2× their respective MIC) resulted in improved antibacterial activity compared with amikacin or meropenem alone, respectively (FIG. 28), thereby confirming the results of Example 4.

Example 6

### Mouse Model of A. baumannii Infection

The aim of this study was to investigate the efficacy of co-administration of compound (I) with amikacin, meropenem or colistin, in a mouse model of A. baumannii pneumonia infected via the intra-tracheal route.

Materials

Compound (I) was supplied as a nanosuspension, prepared by wet milling as follows. The milling medium was prepared by dissolving a polymer, PVP (polyvinylpyrrolidone)12 PF (Kollidon 12 PF, BASF), at 50.0 mg/mL in purified water at 50 mL scale. The solution was added to a 250 mL clear glass vial (Type II), followed by addition of compound (I) at 100 mg/mL. To avoid aggregation, immediately after compounding a pre-suspension was made by stirring using a magnetic stirrer for 30 minutes. Finally, the milling beads were added. 225 g of ytrium stabilized zirconium oxide beads with a diameter of 0.5 mm were added. The vial was closed and placed on a roller mill (Peira, Beerse, Belgium) for the wet milling process. The vial speed was set at 160 rpm (setting 600). After 64 hr of wet milling a sample was taken for analysis of particle size distribution using laser diffraction, type Mastersizer 2000 (Malvern, Worcestershire, UK) to confirm that at least 90% of the suspended particles were inferior to 500 nm in diameter and the milling process was stopped. The white homogeneous nanosuspension was then harvested from the milling beads using a syringe with a 23 G needle.

The particular composition of the nanosuspension used in each study is described in the relevant “Study design” section.

Two strains of A. baumannii were used.

SAN-94040 (SAN): cephalosporinase-overproducing strain, susceptible to meropenem and intermediate to amikacin and resistant to fluoroquinolones, isolated from blood cultures of an intensive care patient with nosocomial pneumonia from North Africa (Algeria). The SAN strain is responsible for an average of 80% mortality rate in this mice pneumonia model.

RCH-69: cephalosporinase producing strain; low susceptibility to imipenem and resistant to amikacin.

Test System and Infection Establishment

The bacterial inoculum was prepared in order to obtain a suspension containing 10⁸ CFU/mL of Acinetobacter baumannii in saline.

Experimentations were performed with six-week old female C3H/HeN mice (18-20 g, Breeding Centre JANVIER (Ile saint Genest Mayenne 53)) because of their particular susceptibility to Acinetobacter baumannii and the reproducibility of the results (inbreeding in the breed). Each mouse was individually marked and housed in cages containing five animals/cage under ventilated and controlled (temperature and humidity) atmosphere.

The mice were rendered transiently neutropenic by 2 intra-peritoneal (IP) injections of 150 μL of cyclophosphamide (Endoxan® 150 mg/kg) 4 (D-4) and 3 (D-3) days before the Acinetobacter baumannii inoculation (Day 0). This transient immunosuppression was sufficient to facilitate the development of the A. baumannii pneumonia while allowing the recovery of the neutrophil influx 2 (Day 2) days after inoculation in order to avoid or limit a systemic bacterial dissemination.

Mice were anesthetized by a mixture of oxygen and isoflurane. The bacterial inoculation was performed on Day 0 by intra-tracheal installation using an endotracheal catheter (50 μl of the suspension, thus 5.106 CFU). The positioning of the catheter in the trachea (instead of in the oesophagus) was checked by a “ring test”. The bacterial count in lungs was checked for all study groups by sacrificing one mouse immediately after inoculation.

In Vivo End Points

Survival Rate

Survival observations were assessed twice daily, up to 4 days post infection. The survival rates were compared with the Chi-square test or the Fisher exact test as appropriate. The survival curves were compared by the Kaplan-Meier method.

Bacterial Count in Blood and Tissues

Sample time points: T0 (pre-inoculation), T24h (post inoculation), T48h and T72h.

Blood samples preparation: At each time point, three mice were sacrificed per group by cervical dislocation.

Tissue samples preparation: At each time point, three mice were sacrificed per group. Lungs, kidneys, liver and spleen were collected after the blood sampling and cervical dislocation. Tissues were wiped with a sterile gauze to remove blood contaminant outside. Tissues were then be weighed, finely ground and plated on agar for quantitative culture. Cultures were incubated at 37° C. for 18 to 24 hours.

Statistical Analysis

Student's t test, X₂ or Kruskal Wallis test was used to compare the CFU counts. The survival rates were be compared with the Chi-square test or the Fisher exact test as appropriate.

The survival curves were compared by the Kaplan-Meier method.

P<0.05 was be considered statistically significant.

Three studies were carried out as follows:

Study 1

The aim of this experiment was to assess the survival rate of transiently neutropenic female C3H/HeN mice administered, 3 hours post-inoculation of A. baumannii SAN-94040 via intra-tracheal route, with compound (I) via intra-peritoneal route at 200 mg/kg 3 times daily (every 8 hours) for 1 day alone and in combination with meropenem at 50 mg/kg 3 times daily (every 8 hours) for 1 day or with amikacin at 18 mg/kg 3 times daily (every 8 hours) for 1 day. 84 mice were allocated into 6 groups as shown in Table 10.

The SAN-94040 strain of A. baumannii is susceptible to meropenem and has intermediate susceptibility to amikacin.

TABLE 10
Study 1 design

| | | | Com- | Dose and | |
|---|---|---|---|---|---|
| Group | N* | Route | pound | Regimen | Volume |
| 1 | 14 | I.P. | 0.9% NaCl | Q8h for 1 Day | 200 μL |
| 2 | 14 | I.P. | compound | 200 mg/kg Q8h | 200 μL of |
| | | | (I) | for 1 Day as a | the 20 mg/mL |
| | | | | bolus | suspension |
| 3 | 14 | I.P. | amikacin | 18 mg/kg once | 200 μL |
| | | | | as a bolus | |
| 4 | 14 | I.P. | meropenem | 50 mg/kg Q8h for | 200 μL |
| | | | | 1 Day as a bolus | |
| 5 | 14 | I.P. | compound | compound (I): | compound (I): |
| | | | (I) + | 200 mg/kg Q8h | 100 μL of |
| | | | amikacin | for 1 Day | the 40 mg/mL |
| | | | | amikacin: 18 | suspension |
| | | | | mg/kg once | amikacin: |
| | | | | | 100 μL |
| 6 | 14 | I.P. | compound | compound (I): | compound (I): |
| | | | (I) + | 200 mg/kg Q8h | 100 μL of |
| | | | meropenem | for 1 Day | the 40 mg/mL |
| | | | | meropenem: 50 | suspension |
| | | | | mg/kg Q8h for 1 | meropenem: |
| | | | | Day | 100 μL |

*number of mice

Three vials of 8 mL each containing 91.6 mg of compound (I)/mL and 45.8 mg of PVP 12 PF (Kollidon 12 PF)/mL were prepared and stored between +02° C. and +08° C. Following storage, each vial was homogenized by manual shaking and diluted by 2-fold in 16.17 mg/mL NaCl aqueous solution in order to obtain a 45.8 mg of compound (I)/mL isotonic suspension.

In Group 2 (compound (I) alone), mice were administered with 200 μL of a 20 mg/mL compound (I) suspension obtained by diluting 2.183 mL of the 45.8 mg/mL suspension in 2.817 mL of 0.9% NaCl aqueous solution.

In Groups 5 and 6 (compound (I) in combination with amikacin and meropenem, respectively), mice were administered with 100 μL of a 40 mg/mL compound (I) suspension obtained by diluting 4.367 mL of the 45.8 mg/mL suspension in 0.683 mL of 0.9% NaCl aqueous solution.

Meropenem was supplied by ASTRA ZENECA (Meronem®) (50 mg of meropenem/mL). Diluted solution was used extemporaneously after dilution in 0.9% NaCl.

Amikacin was supplied by MYLAN LAB (Amiklin®) (50 mg of amikacin/mL). Diluted solution was used extemporaneously after dilution in 0.9% NaCl.

Results—Survival

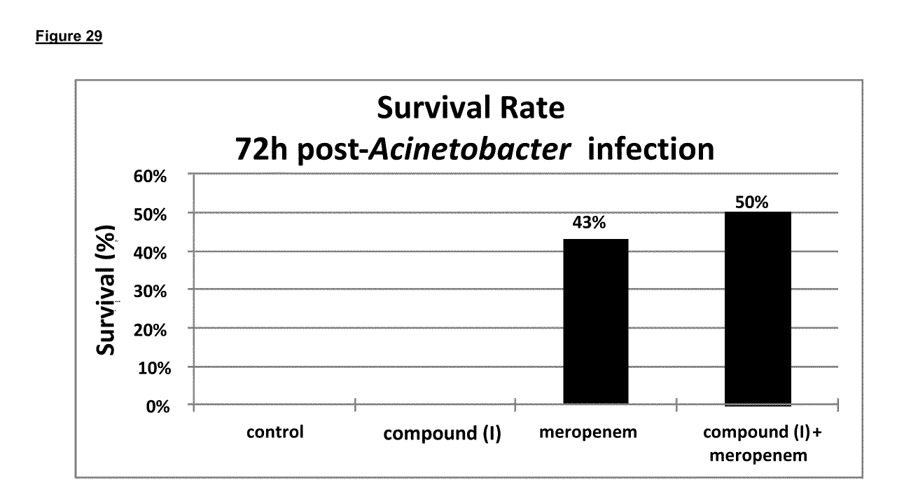

Results are presented in Table 11 and FIG. 29.

TABLE 11
Effect of antibacterial agent therapy on survival
No. of survivors (%)

| Time after | control | compound (I) | amikacin | meropenem | | |
|---|---|---|---|---|---|---|
| SAN-94040 | 0.9% NaCl | 200 mg/kg | 18 mg/kg | 50 mg/kg | compound (I) + | compound (I) + |
| strain | q 8 h I.P. | q 8 h I.P. | q 8 h I.P. | q 8 h I.P. | amikacin | meropenem |
| infection (h) | (n = 14) | (n = 14) | (n = 14) | (n = 14) | (n = 14) | (n = 7) |
| 0 | 14(100) | 14(100) | 14(100) | 14(100) | 14(100) | 14(100) |
| 24 | 7(50) | 10(71) | 12(86) | 14(100) | 13(93) | 14(100) |
| 48 | 0 | 0 | 1(17) | 11(79) | 3(21) | 8(57) |
| 72 | 0 | 0 | 0 | 6(43) | 0 | 7(50) |
| 96 | 0 | 0 | 0 | 6(43) | 0 | 5(36) |

All animals of the control group died within 48 hours post-infection as expected in this model of acute pneumonia.

The mice administered with compound (I) at 200 mg/kg Q8h for 1 day died also within 48 hours post-infection.

Treatments using meropenem alone or meropenem+compound (I) demonstrated significant efficacy compared to the control group. The combination of compound (I)+meropenem demonstrated increased survival rate (50%) compared to meropenem alone (43%) or compound (I) alone (0%), 72 hours post-infection (FIG. 29).

The SAN-94040 strain of A. baumannii has intermediate susceptibility to amikacin, and since amikacin does not penetrate very well into the lungs, it was not unexpected that all animals died within 72 hours post-infection when treated with amikacin alone or with amikacin+compound (I).

Study 2

The aim of this experiment was to assess the bacterial counts in blood, lungs, spleen, liver and kidneys of transiently neutropenic female C3H/HeN mice administered, 3 hours post-inoculation of A. baumannii SAN-94040 via intra-tracheal route, with compound (I) via intra-peritoneal route at 200 mg/kg 3 times daily (every 8 hours) for 1 day alone and in combination with meropenem at 50 mg/kg 3 times daily (every 8 hours) for 1 day or with amikacin at 18 mg/kg 3 times daily (every 8 hours) for 1 day. 90 mice were allocated into 6 groups shown in Table 12.

The SAN-94040 strain of A. baumannii is susceptible to meropenem and has intermediate susceptibility to amikacin.

TABLE 12
Study 2 design
The test item administration via intra-peritoneal route was initiated 3
hours after inoculation. 90 mice were allocated into 6 groups as follow:

| | | | Com- | Dose and | |
|---|---|---|---|---|---|
| Group | N* | Route | pound | Regimen | Volume |
| 1 | 15 | I.P. | 0.9% NaCl | Q8h for 1 Day | 200 μL |
| 2 | 15 | I.P. | compound | 200 mg/kg Q8h | 100 μL of |
| | | | (I) | for 1 Day as | the 46.5 mg/mL |
| | | | | a bolus | suspension + |
| | | | | | 100 μL 0.9% |
| | | | | | NaCl |
| 3 | 15 | I.P. | amikacin | 18 mg/kg once | 200 μL |
| | | | | as a bolus | |
| 4 | 15 | I.P. | meropenem | 50 mg/kg Q8h | 200 μL |
| | | | | for 1 Day as | |
| | | | | a bolus | |
| 5 | 15 | I.P. | compound | compound (I): | compound (I): |
| | | | (I) + | 200 mg/kg Q8h | 100 μL of |
| | | | amikacin | for 1 Day | the 46.5 mg/mL |
| | | | | amikacin: | suspension + |
| | | | | 18 mg/kg once | amikacin: |
| | | | | | (100 μL) |
| 6 | 15 | I.P. | compound | compound (I): | compound (I): |
| | | | (I) + | 200 mg/kg Q8h | 100 μL of |
| | | | meropenem | for 1 Day | the 46.5 mg/mL |
| | | | | meropenem: 50 | suspension + |
| | | | | mg/kg Q8h | meropenem |
| | | | | for 1 Day | (100 μL) |

*number of mice

Two vials of 8 mL each containing 93.1 mg of compound (I) and 46.6 mg of PVP 12 PF (Kollidon 12 PF)/mL were prepared and stored between +02° C. and +08° C. Following storage, each vial was homogenized by manual shaking and diluted by 2-fold in 16.17 mg/mL NaCl aqueous solution in order to obtain a 46.5 mg of compound (I)/mL isotonic suspension.

In Groups 2, 5 and 6, mice were administered with 100 μL of the 46.5 mg/mL compound (I) suspension.

Meropenem supplied by ASTRA ZENECA (Meronem®) (50 mg of meropenem/mL). Diluted solution was used extemporaneously after dilution in 0.9% NaCl.

Amikacin supplied by MYLAN LAB (Amiklin®) (50 mg of amikacin/mL). Diluted solution was used extemporaneously after dilution in 0.9% NaCl.

Results—Bacterial Cell Counts in the Lungs

All results concerning bacterial counts were expressed from Day 0 (inoculation) to 72 hours post-inoculation (Day 3). Bacterial counts on Day 3 were carried out only on surviving mice and results were more objectionable for interpretation. On Day 0, only bacterial count in lung of control mice was performed. Bacterial cell counts were determined as set out in the General Procedures. The results are summarised in Table 13.

TABLE 13
Results of lung bacterial counts of A. baumannii

| CFU/g Lung | T0 | 24 h | sd* | 48 h | sd* | 72 h | sd* |
|---|---|---|---|---|---|---|---|
| control | 3.33E+06 | 1.81E+10 | 1.58E+10 | 1.82E+10 | 7.91E+09 | 2.50E+02 | 1.00E+00 |
| compound (I) | | 4.72E+09 | 4.99E+09 | 3.75E+05 | 1.00E+00 | | |
| amikacin | | 1.33E+09 | 2.10E+09 | 4.41E+08 | 6.24E+08 | | |
| meropenem | | 3.75E+04 | 4.90E+04 | 5.56E+06 | 7.86E+06 | 1.00E+00 | 1.00E+00 |
| compound (I) + | | 6.86E+08 | 1.19E+09 | 1.31E+09 | 7.95E+08 | 3.57E+02 | 1.00E+00 |
| amikacin | | | | | | | |
| compound (I) + | | 3.22E+03 | 2.56E+03 | 1.18E+09 | 1.66E+09 | 5.85E+05 | 7.61E+04 |
| meropenem | | | | | | | |

*standard deviation

At 24 h and 48 h after inoculation, control mice had positive lung cultures, reflecting the virulence of the infection model. At 24 h after inoculation, administration of a combination of compound (I) with meropenem reduced bacterial counts in lungs compared to control, and the reduction was greater than with administration of compound (I) or meropenem alone. At 24 h after inoculation, administration of a combination of compound (I) with amikacin reduced bacterial counts in lungs compared to control, and the reduction was greater than with administration of compound (I) or amikacin alone.

Results—Bacterial Cell Counts in the Blood

Bacterial cell counts were determined as set out in the General Procedures. The results are summarised in Table 14.

TABLE 14
Results of blood bacterial counts of A. baumannii

| CFU/mL Blood | T0 | 24 h | sd* | 48 h | sd* | 72 h | sd* |
|---|---|---|---|---|---|---|---|
| Control | — | 1.5 10⁶ | 2.2 10⁶ | 3 10⁶ | 4 10⁶ | 0 | 0 |
| compound (I) | — | 2.4 10⁶ | 4 10⁶ | 3 10² | 0 | — | — |
| amikacin | — | 1.3 10⁷ | 1.5 10⁷ | 1.6 10⁴ | 1.9 10⁴ | — | — |
| meropenem | — | 0 | 0 | 0 | 0 | 0 | 0 |
| compound (I) + | — | 1.4 10³ | 2 10³ | 7.5 10⁶ | 1 10⁷ | — | — |
| amikacin | | | | | | | |
| compound (I) + | — | 7.3 10² | 1.3 10³ | 0 | 0 | 0 | 0 |
| meropenem | | | | | | | |

*standard deviation

At 24 h after inoculation, administration of a combination of compound (I) and amikacin reduced bacterial blood counts compared to control, and the reduction was greater than with administration of compound (I) or amikacin alone. At 48 h after inoculation, administration of a combination of compound (I) and meropenem reduced the bacterial blood counts compared to control and reduced the bacterial blood count to zero.

Results—Bacterial Cell Counts in the Spleen

Bacterial cell counts were determined as set out in the General Procedures. The results are summarised in Table 15.

TABLE 15
Results of spleen bacterial counts of A. baumannii

| CFU/g Spleen | T0 | 24 h | sd* | 48 h | sd* | 72 h | sd* |
|---|---|---|---|---|---|---|---|
| Control | — | 2.5 10⁸ | 4.3 10⁸ | 1.7 10⁸ | 2.5 10⁸ | 0 | 0 |
| compound (I) | — | 7.2 10⁸ | 1.2 10⁹ | 2.4 10⁵ | 0 | — | — |
| Amikacin | — | 3.7 10⁸ | 5.7 10⁸ | 6.8 10⁶ | 9.5 10⁶ | — | — |
| meropenem | — | 2.4 10⁴ | 1.9 10⁴ | 1.25 10³ | 1.7 10³ | 0 | 0 |
| compound (I) + | — | 2.3 10⁶ | 4 10⁶ | 2.3 10⁴ | 2.8 10⁴ | 0 | 0 |
| amikacin | | | | | | | |
| compound (I) + | — | 3.7 10⁵ | 3.7 10⁵ | 1.7 10⁵ | 1.9 10⁵ | 8.3 10² | 1.2 10³ |
| meropenem | | | | | | | |

*standard deviation

At 24 h and 48 h after inoculation, administration of a combination of compound (I) and amikacin reduced bacterial spleen counts compared to control, and the reduction was greater than with administration of compound (I) or amikacin alone. At 24 h and 48 h after inoculation, administration of a combination of compound (I) and meropenem reduced bacterial spleen counts compared to control and the reduction was greater than with administration of compound (I) alone.

Results—Bacterial Cell Counts in the Liver Bacterial cell counts were determined as set out in the General Procedures.

TABLE 16
Results of liver bacterial counts of Acinetobacter baumannii

| CFU/g Liver | T0 | 24 h | sd* | 48 h | sd* | 72 h | sd* |
|---|---|---|---|---|---|---|---|
| Control | — | 1.46 10⁸ | 1.75 10⁸ | 1.12 10⁷ | 1.6 10⁷ | 0 | 0 |
| compound (I) | — | 1.05 10⁸ | 1.8 10⁸ | 2.4 10⁵ | 0 | — | — |
| amikacin | — | 1.03 10⁷ | 1.75 10⁷ | 1.8 10⁵ | 1.4 10⁵ | — | — |
| meropenem | — | 1.75 10⁵ | 2.8 10⁵ | 0 | 0 | 0 | 0 |
| compound (I) + | — | 1.3 10⁵ | 2.11 10⁵ | 6.6 10⁴ | 9.3 10⁴ | 0 | 0 |
| amikacin | | | | | | | |
| compound (I) + | — | 1.1 10⁵ | 1.20 10⁵ | 8.4 10³ | 1.16 10⁴ | 0 | 0 |
| meropenem | | | | | | | |

*standard deviation

At 24 h and 48 h after inoculation, administration of a combination of compound (I) and amikacin reduced bacterial liver counts compared to control, and the reduction was greater than with administration of compound (I) or amikacin alone. At 24 h and 48 h after inoculation, administration of a combination of compound (I) and meropenem reduced bacterial liver counts compared to control and the reduction was greater than with administration of compound (I) alone.

Results—Bacterial Cell Counts in the Kidneys

TABLE 17
Results of kidney bacterial counts of Acinetobacter baumannii

| CFU/g Kidneys | T0 | 24 h | sd* | 48 h | sd* | 72 h | sd* |
|---|---|---|---|---|---|---|---|
| Control | — | 6.35 10⁸ | 6.4 10⁸ | 3.4 10⁷ | 4.4 10⁷ | 0 | 0 |
| compound (I) | — | 2.36 10⁸ | 3.6 10⁸ | 2.4 10⁵ | 0 | — | — |
| amikacin | — | 1.09 10⁷ | 1.8 10⁷ | 6.8 10⁵ | 9.7 10⁵ | — | — |
| meropenem | — | 1.34 10⁴ | 1.06 10⁴ | 0 | 0 | 0 | 0 |
| compound (I) + | — | 1.28 10⁵ | 2.2 10⁵ | 1.35 10⁶ | 4.9 10⁵ | 0 | 0 |
| amikacin | | | | | | | |
| compound (I) + | — | 4.31 10² | 4.5 10² | 4.7 10⁵ | 6.7 10⁵ | 0 | 0 |
| meropenem | | | | | | | |

*standard deviation

At 24 h after inoculation, administration of a combination of compound (I) and amikacin reduced bacterial kidney counts compared to control, and the reduction was greater than with administration of compound (I) or amikacin alone. At 24 h after inoculation, administration of a combination of compound (I) and meropenem reduced bacterial kidney counts compared to control, and the reduction was greater that with administration of compound (I) alone or meropenem alone.

Study 3

The aim of this experiment was to assess the survival rate of transiently neutropenic female C3H/HeN mice administered, 3 hours post-inoculation of A. baumannii RCH-69 via intra-tracheal route, with a compound (I) via intra-peritoneal route at 200 mg/kg 3 times daily (every 8 hours) for 3 days alone and in combination with meropenem at 50 mg/kg 3 times daily (every 8 hours) for 3 days or with colistin at 125000 UI/kg 3 times daily (every 8 hours) for 3 days. 85 mice were allocated into 6 groups as follows.

The RCH-69 strain of A. baumannii has low susceptibility to imipenem, is assumed to have a low susceptibility to meropenem and is resistant to amikacin.

TABLE 18
Study 3 design
The test item administration via intra-peritoneal route was initiated 3
hours after inoculation. 85 mice were allocated into 6 groups as follow:

| | | | Com- | Dose and | |
|---|---|---|---|---|---|
| Group | N* | Route | pound | Regimen | Volume |
| 1 | 13 | I.P. | 0.9% NaCl | Q8h for 3 Days | 200 μL |
| 2 | 13 | I.P. | compound | 200 mg/kg Q8h | 100 μL of |
| | | | (I) | for 3 Days as | the 47.1 mg/mL |
| | | | | a bolus | suspension + |
| | | | | | 100 μL 0.9% |
| | | | | | NaCl |
| 3 | 14 | I.P. | meropenem | 50 mg/kg Q8h | 200 μL |
| | | | | for 3 Days as | |
| | | | | a bolus | |
| 4 | 15 | I.P. | compound | compound (I): | compound (I): |
| | | | (I) + | 200 mg/kg Q8h | 100 μL of |
| | | | meropenem | for 3 Days + | the 47.1 mg/mL |
| | | | | meropenem: 50 | suspension + |
| | | | | mg/kg Q8h for | meropenem: |
| | | | | 3 Days | (100 μL) |
| 5 | 15 | I.P. | compound | compound (I): | compound (I): |
| | | | (I) + | 200 mg/kg Q8h | 100 μL of |
| | | | colistin | for 3 Days + | the 47.1 mg/mL |
| | | | | colistin: | suspension + |
| | | | | 125000 U/kg | colistin: |
| | | | | Q8h for | (100 μL) |
| | | | | 3 Days | |
| 6 | 15 | I.P. | meropenem + | meropenem: | meropenem: |
| | | | colistin | 50 mg/kg Q8h | (100 μL) + |
| | | | | for 3 Days + | colistin: |
| | | | | colistin: | (100 μL) |
| | | | | 125000 U/kg | |
| | | | | Q8h for | |
| | | | | 3 Days | |

*number of mice

Six vials of 8 mL each containing 94.2 mg/mL of compound (I) and 47.1 mg/mL of PVP 12 PF (Kollidon 12 PF) were prepared and stored between +2° C. and +8° C. Following storage, each vial was homogenized by manual shaking and diluted by 2-fold in 13.72 mg/mL NaCl aqueous solution in order to obtain a 47.1 mg of compound (I)/mL isotonic suspension. In Groups 2, 4 and 5, mice were administered with 100 μL of the 47.1 mg/mL compound (I) suspension.

Results—Survival

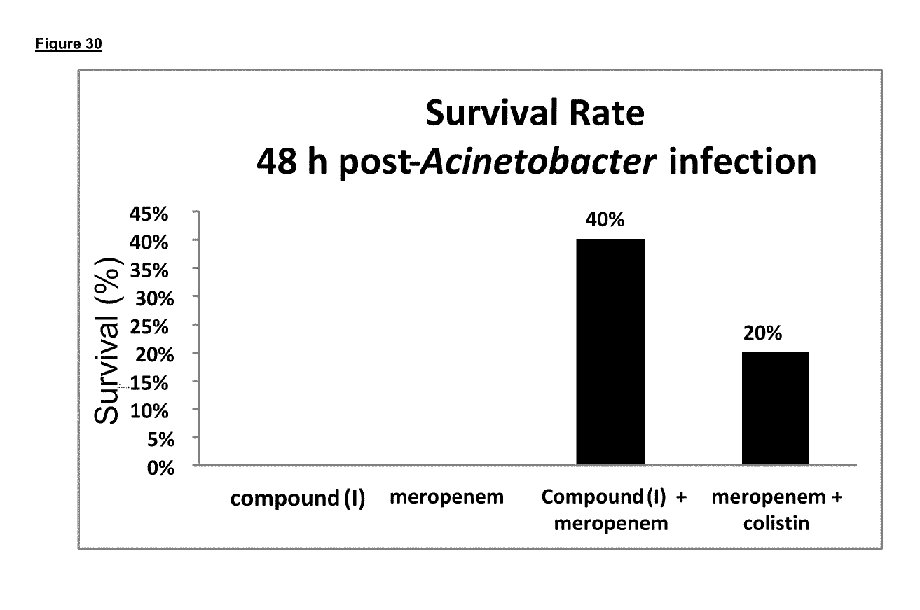

Results are presented in Table 19 and FIG. 30.

TABLE 19
Effect of antibacterial agent therapy on survival
No. of survivors (%)

| | | | | compound (I) + | compound (I) + | meropenem + |
|---|---|---|---|---|---|---|
| | control 0.9% | compound (I) | meropenem | meropenem 200 mg/kg + | colistin 200 mg/kg + | colistin 50 mg/kg + |
| Time after | NaCl q 8 h | 200 mg/kg q 8 h | 50 mg/kg q 8 h | 50 mg/kg | 125000 Ul/kg | 125000 Ul/kg |
| RCH strain | I.P. | I.P. 3 days | I.P. 3 days | q 8 h I.P. 3 days | q 8 h I.P. 3 days | q 8 h I.P. 3 days |
| infection (h) | (n = 13) | (n = 13) | (n = 14) | (n = 15) | (n = 15) | (n = 15) |
| 0 | 13(100) | 13(100) | 14(100) | 15(100) | 15(100) | 15(100) |
| 24 | 2(15) | 5(38) | 14(100) | 15(100) | 15(100) | 14(93) |
| 48 | 0 | 0 | 0 | 6(40) | 0 | 3(20) |
| 72 | 0 | 0 | 0 | 0 | 0 | 1(7) |
| 96 | 0 | 0 | 0 | 0 | 0 | 0 |

All animals of the control group died within 48 hours following the inoculation of Acinetobacter baumannii RCH-69 (intermediate to imipenem and susceptible to colistin) as expected in this model of acute pneumonia. The mice administered with compound (I) at 200 mg/kg Q8 h for 3 days died within 48 hours post-infection and the mice administered with meropenem at 50 mg/kg Q8 h for 3 days died also within 48 hours post-infection as expected as RCH-69 strain has a low susceptibility to carbapenems.

Treatments using a combination of compound (I) and meropenem or a combination of meropenem and colistin exhibited significant efficacy compared to the control group or compound (I) alone or meropenem alone. A combination of compound (I) and meropenem demonstrated increased survival rate (40%) compared to a combination of meropenem and colistin (20%) at 48 hours post-infection (FIG. 30).

### Example 7

### Additional Mouse Model of A. baumannii Infection

The aim of this study was to consolidate the results of Example 6 regarding the efficacy of compound (I) administered alone, and of co-administration of compound (I) with meropenem in a mouse model of A. baumannii pneumonia infected via the intra-tracheal route.

Materials

Compound (I) was supplied as a nanosuspension, prepared by wet milling as described in Example 6. The particular composition of the nanosuspension used in the study is described in the “Study design” section.

The following strain of A. baumannii was used:

SAN-94040 (SAN): cephalosporinase-overproducing strain, susceptible to meropenem and intermediate to amikacin and resistant to fluoroquinolones, isolated from blood cultures of an intensive care patient with nosocomial pneumonia from North Africa (Algeria). The SAN strain is responsible for an average of 80% mortality rate in this mice pneumonia model.

Test System and Infection Establishment

The bacterial inoculum was prepared in order to obtain a suspension containing 10⁸ CFU/mL of Acinetobacter baumannii in saline, unless indicated otherwise.

Experimentations were performed with six-week old female C3H/HeN mice (18-20 g, Harlan Laboratories) because of their particular susceptibility to Acinetobacter baumannii. Each mouse was individually marked and housed in cages containing five animals/cage under ventilated and controlled (temperature and humidity) atmosphere.

The mice were rendered transiently neutropenic by 2 intra-peritoneal (IP) injections of 150 μL of cyclophosphamide (150 mg/kg) 4 (D-4) and 3 (D-3) days before the Acinetobacter baumannii inoculation (Day 0).

Mice were infected with 50 μL the prepared bacterial suspension (inoculums depending on experiment) via intra-tracheal inoculation of mouse. In the study, three animals (Group 1) were sacrificed after inoculation to determine bacterial burden in lungs at the time of infection. Lungs were aseptically removed, weighed and homogenized in 1 mL of 0.9% NaCl. Homogenates were serially diluted and plated on TSA agar. Plates were incubated overnight at 37° C. in 5% CO₂. Following incubation, colonies were counted for bacterial growth.

In vivo end points:

Survival Rate

Animal survival was assessed every day for 7 days. Survival data were tabulated and a Probit analysis performed to determine the 50% protective dose (PD₅₀) for each group.

The study was carried out as follows:

Study

The aim of this experiment was to assess the survival rate of transiently neutropenic female C3H/HeN mice administered, 3 hours post-inoculation of A. baumannii SAN-94040 via intra-tracheal route, with compound (I) via intra-peritoneal route at 200 mg/kg 3 times daily (every 8 hours) for 1 day alone and in combination with meropenem at 30 mg/kg 3 times daily (every 8 hours) for 1 day. 53 mice were allocated into 6 groups as shown in Table 20.

TABLE 20
Study design

| | | | | Dose and |
|---|---|---|---|---|
| Group | N* | Route | Compound | Regimen |
| 1 | 3 | N/A | pre-treatment | N/A |
| | | | control | |
| 2 | 10 | I.P. | control | Q8h, 1 day |
| | | | (0.9% NaCl) | |
| 3 | 10 | I.P. | compound (I) | 200 mg/kg Q8h |
| | | | | for 1 day |
| 4 | 10 | I.P. | meropenem | 30 mg/kg Q8 |
| | | | | for 1 day |
| 5 | 10 | I.P. | compound | compound (I): |
| | | | (I) + | 200 mg/kg Q8h |
| | | | meropenem | for 1 day + |
| | | | | meropenem: |
| | | | | 30 mg/kg Q8h |
| | | | | for 1 day |
| 6 | 10 | I.P. | meropenem + | meropenem: |
| | | | colistin | 30 mg/kg Q8h |
| | | | | for 1 day + |
| | | | | colistin: |
| | | | | 125000 UI/kg Q8h |
| | | | | for 1 day |

*number of mice

Mice were dosed via intraperitoneal injection into the tail vein according to the schedule below.

In Group 2 (control) mice were administered with 100 μL of 0.9% NaCl solution (Teknova).

Five vials of 5 mL each containing 94 mg of compound (I)/mL and 47 mg of PVP 12 PF (Kollidon 12 PF)/mL (compound (I) nanosuspension) were prepared and stored between +2° C. and +8° C. Following storage, each vial was homogenized by manual shaking and diluted 25/1 (v/v) in 193.76 mg/mL NaCl aqueous solution in order to obtain a 90.4 mg of compound (I)/mL isotonic suspension. Diluted suspension was stable for 24 hours between +2° C. and +8° C. Before use, diluted compound (I) nanosuspension was homogenized by manual shaking (2 movements/second) for 60 seconds.

In Group 3 (compound (I) alone at 200 mg/kg/administration), mice were dosed first with 2.21 mL/kg/administration (around 45 μL/mouse) of the 90.4 mg/mL compound (I) diluted isotonic nanosuspension and then with 55 μL of NaCl 0.9% solution.

In Group 4 (meropenem alone at 30 mg/kg/administration), mice were dosed first with 1.65 mL/kg/administration (around 55 μL/mouse) of the 18.2 mg/mL meropenem solution & then with 45 μL of NaCl 0.9% solution

In Group 5 (compound (I) at 200 mg/kg/administration+meropenem at 30 mg/kg/administration), mice were dosed first with 2.21 mL/kg/administration (around 45 μL/mouse) of the 90.4 mg/mL compound (I) diluted isotonic nanosuspension & then with 1.65 mL/kg/administration (around 55 μL/mouse) of the 18.2 mg/mL meropenem solution

Meropenem (USP reference Standard) was prepared as follows: 182 mg of Meropenem was reconstituted with 10 mL of sterile water for injection to achieve a concentration of 18.2 mg/mL. The solution was vortexed until the meropenem dissolved.

Results—Survival

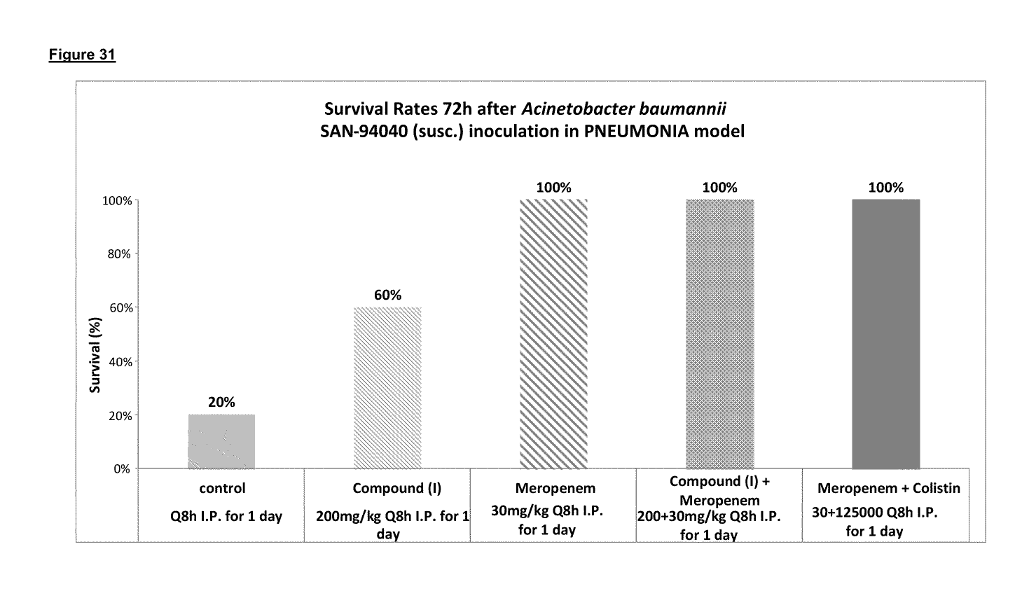

Results are presented in Table 21 and FIG. 31.

TABLE 21
Effect of antibacterial agent therapy on survival
No. of survivors (%)

| | | | | compound (I) + | meropenem + |
|---|---|---|---|---|---|
| | control | compound (I) | meropenem | meropenem 200 mg/kg + | colistin 30 mg/kg + |
| Time after | 0.9% NaCl Q8h | 200 mg/kg Q8h | 30 mg/kg Q8h | 30 mg/kg Q8h | 125000 UI/kg Q8h |
| AB SAN-94040 | I.P. for 1 day | I.P. for 1 day | I.P. for 1 day | I.P. for 1 day | I.P. for 1 day |
| infection (h) | (n = 10) | (n = 10) | (n = 10) | (n = 10) | (n = 10) |
| 24 | 10 (100%) | 10 (100%) | 10 (100%) | 10 (100%) | 10 (100%) |
| 48 | 10 (100%) | 8 (80%) | 10 (100%) | 10 (100%) | 10 (100%) |
| 72 | 2 (20%) | 6 (60%) | 10 (100%) | 10 (100%) | 10 (100%) |
| 96 | 2 (20%) | 6 (60%) | 10 (100%) | 10 (100%) | 10 (100%) |

In the control group, 80% of mice died within 72 hours following the inoculation of Acinetobacter baumannii SAN-94040 isolate as expected in this model of acute pneumonia. Treatments using I.P. administration of compound (I) as monotherapy demonstrated improved survival rate (60%) and a combination of compound (I) and meropenem exhibited significant efficacy (100%) compared to the control group or compound (I) alone.

Conclusion—In Vivo Studies of Examples 6 and 7

In the survival study, combinations of compound (I) with meropenem and compound (I) with amikacin demonstrated increased survival rates compared to administration of meropenem or amikacin alone, with the compound (I) with meropenem combination being particularly effective. A combination of compound (I) with colistin was shown to exhibit greater efficacy than administration of compound (I) alone.

Bacterial cell count studies indicated that, depending on the organ/system in question, at different time points after administration the combinations of compound (I) with meropenem and compound (I) with amikacin led to a lower bacterial count than administration of any as a monotherapy.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

Patents and patent applications referred to herein are herein incorporated by reference in their entirety.

1. A method of treating microbial infection comprising administering to a subject in need thereof a therapeutically effective amount of:
(a) 4-(4-ethyl-5-fluoro-2-hydroxyphenoxy)-3-fluorobenzamide; and
(b) an antibacterial agent selected from the group consisting of carbapenems, aminoglycosides, polymixins, glycylcyclines, rifampicin and sulbactam.

2. A method according to claim 1, wherein the antibacterial agent is a carbapenem and is selected from the group consisting of meropenem, imipenem, ertapenem, doripenem, panipenem and biapenem, suitably meropenem and imipenem.

3. A method according to claim 2, wherein the antibacterial agent is meropenem.

4. A method according to claim 1, wherein the antibacterial agent is an aminoglycoside and is selected from the group consisting of amikacin, gentamicin, tobramycin, arbekacin, kanamycin, neomycin, netilmicin, paromomycin, rhodostreptomycin, streptomycin and apramycin, suitably amikacin, gentamicin and tobramycin.

5. A method according to claim 4, wherein the antibacterial agent is amikacin.

6. A method according to claim 1, wherein the antibacterial agent is a polymixin and is selected from the group consisting of colistimethate sodium and colistin sulfate.

7. A method according to claim 6, wherein the antibacterial agent is colistimethate sodium.

8. A method according to claim 1, wherein the antibacterial agent is a glycylcycline and is tigecycline.

9. A method according to claim 1, wherein the antibacterial agent is rifampicin.

10. A method according to claim 1, wherein the antibacterial agent is sulbactam.

11. A method according to claim 1, wherein the microbial infection is a human or animal infection by Acinetobacter.

12. A method according to claim 11, wherein the microbial infection is a human or animal infection by Acinetobacter baumannii.

13. A method according to claim 11, wherein the microbial infection is associated with a disease or disorder selected from the group consisting of a blood stream infection, a urinary tract infection, meningitis, a wound infection (especially in a burns patient) and pneumonia (especially in a patient on mechanical ventilation).

14. A method according to claim 1, wherein said 4-(4-ethyl-5-fluoro-2-hydroxyphenoxy)-3-fluorobenzamide and said antibacterial agent are both administered intravenously.

15. A method according to claim 14, wherein said 4-(4-ethyl-5-fluoro-2-hydroxyphenoxy)-3-fluorobenzamide and said antibacterial agent are administered simultaneously.

16. A method according to claim 14, wherein said 4-(4-ethyl-5-fluoro-2-hydroxyphenoxy)-3-fluorobenzamide and said antibacterial agent are administered sequentially.

17. A method according to claim 16, wherein said 4-(4-ethyl-5-fluoro-2-hydroxyphenoxy)-3-fluorobenzamide is administered prior to said antibacterial agent.

18. A method according to claim 16, wherein said antibacterial agent is administered prior to said 4-(4-ethyl-5-fluoro-2-hydroxyphenoxy)-3-fluorobenzamide.

19. A pharmaceutical composition comprising:
(a) 4-(4-ethyl-5-fluoro-2-hydroxyphenoxy)-3-fluorobenzamide; and
(b) an antibacterial agent selected from the group consisting of carbapenems, aminoglycosides, polymixins, glycylcyclines, rifampicin and sulbactam.

20. A pharmaceutical composition according to claim 19, wherein the antibacterial agent is selected from the group consisting of meropenem, imipenem, amikacin, gentamicin, tobramycin, colistin and tigecycline, rifampicin and sulbactam.

21. A pharmaceutical composition according to claim 19, wherein the antibacterial agent is a carbapenem and is selected from the group consisting of meropenem, imipenem, ertapenem, doripenem, panipenem and biapenem, suitably meropenem and imipenem.

22. A pharmaceutical composition according to claim 21, wherein the antibacterial agent is meropenem.

23. A pharmaceutical composition according to claim 19, wherein the antibacterial agent is an aminoglycoside and is selected from the group consisting of amikacin, gentamicin, tobramycin, arbekacin, kanamycin, neomycin, netilmicin, paromomycin, rhodostreptomycin, streptomycin and apramycin, suitably amikacin, gentamicin and tobramycin.

24. A pharmaceutical composition according to claim 23, wherein the antibacterial agent is amikacin.

25. A pharmaceutical composition according to claim 19, wherein the antibacterial agent is a polymixin and is selected from the group consisting of colistimethate sodium and colistin sulfate.

26. A pharmaceutical composition according to claim 25, wherein the antibacterial agent is colistimethate sodium.

27. A pharmaceutical composition according to claim 19, wherein the antibacterial agent is a glycylcycline and is tigecycline.

28. A pharmaceutical composition according to claim 19, wherein the antibacterial agent is rifampicin.

29. A pharmaceutical composition according to claim 19, wherein the antibacterial agent is sulbactam.

30. A kit of parts comprising:
(a) a pharmaceutical composition comprising 4-(4-ethyl-5-fluoro-2-hydroxyphenoxy)-3-fluorobenzamide; and
(b) a pharmaceutical composition comprising an antibacterial agent selected from the group consisting of carbapenems, aminoglycosides, polymixins, glycylcyclines, rifampicin and sulbactam.
